(19) United States
(12) Patent Application Publication
(10) Pub. No.: US 2005/0124618 A1
(43) Pub. Date: Jun. 9, 2005

(54) Methods of modulating angiogenesis and cancer cell proliferation

(76) Inventors: Richard J. Roman, Brookfield, WI (US); Andrew Greene, Waukesha, WI (US); Sandra Amaral, Sao Paulo (BR); Alfonso Guillermo Scicli, Birmingham, MI (US); Stephen L. Brown, La Salle (CA)

Correspondence Address: QUARLES & BRADY LLP, 411 E. WISCONSIN AVENUE, SUITE 2040, MILWAUKEE, WI, 53202-4497, US

(21) Appl. No.: 10/986,695

(22) Filed: Nov. 12, 2004

Related U.S. Application Data
(60) Provisional application No. 60/520,172, filed on Nov. 14, 2003.

(51) Int. Cl. A61K 31/537; A61K 31/496; A61K 31/4192; A61K 31/335

(52) U.S. Cl. 514/237.5; 514/601; 514/254.07; 514/560; 514/359

(57) ABSTRACT

Methods for regulating angiogenesis by modulating the activity of 20-HETE are disclosed. Further disclosed are methods of inhibiting cancer and tumor cell growth by exposing the cancer and tumor cells to 20-HETE inhibitors.

### CROSS-REFERENCE TO RELATED APPLICATIONS

This application claims the benefit of U.S. provisional application 60/520,172, filed on Nov. 14, 2003, which is incorporated by reference in its entirety.

### STATEMENT REGARDING FEDERALLY SPONSORED RESEARCH OR DEVELOPMENT

This invention was made with United States government support awarded by the following agency: NIH EY014385 and HL036279. The United States has certain rights in this invention.

### BACKGROUND OF THE INVENTION

Products derived from metabolism of arachidonic acid have been implicated in the regulation of blood vessels and cell growth. When metabolism of arachidonic acid is catalyzed by cytochrome P450, major products are regio- and stereo-specific epoxyeicosatrienoic acids (EETs), their corresponding dihydroxyeicosatrienoic acids (DHETs), and 20-hydroxyeicosatrienoic acid (20-HETE). Cytochrome P450 can also metabolize arachidonic acid to 16-, 17-, 18-, and 19-HETE. Among all isoforms of CYP450, the major enzymes involved in co-hydroxylation of arachidonic acid to 20-HETE are those of the CYP450 4A (CYP4A) and CYP450 4F (CYP4F) families (Roman R J., Physiol. Rev 82:131-185, 2002).

Physiological angiogenesis is a complex process involving an interplay between cells, extracellular matrix molecules, and soluble factors that culminates in cell migration, proliferation, and tube differentiation of endothelial cells. The process of angiogenesis involves preexisting vessels, which send out capillary sprouts to produce new vessels (Hanahan D et al., Science 277: 48-50, 1997). Several cytokines and growth factors, such as vascular endothelial growth factor (VEGF), basic fibroblast growth factor (bFGF), and epidermal growth factor (EGF), have been established to modulate angiogenesis in vitro and in vivo, and, among these factors, VEGF has been considered the most potent angiogenic inducer (Ferrara N, Am J Physiol Cell Physiol 280: C1358-C1366, 2001). Recent studies have further supported the role of VEGF as an important regulator of angiogenesis in skeletal muscle, because treatment with a VEGF-neutralizing antibody blocked the angiogenic response to electrical stimulation and exercise (Amaral S L et al., Microcirculation 8: 57-67, 2001; and Amaral S L et al., Am J Physiol Heart Circ Physiol281: H1163-H1169, 2001).

Epoxygenase metabolites of arachidonic acid have been implicated in endothelial cell migration and tube formation in cultured cells (Natarajan R et al., Am J Physiol Heart Circ Physiol 273: H2224-H2231, 1997; Natarajan R et al., Proc Natl Acad Sci USA 90: 4947-4951, 1993; and Rieder M J et al., Microvasc Res 49: 180-189, 1995). A recent study provided evidence for the expression of cytochrome P-450A (CYP4A) ω-hydroxylase in skeletal muscle cells and arterioles of rat cremaster muscle (Kunert M P et al., Am J Physiol Heart Circ Physiol 280: H1840-H1845, 2001). It has been shown that 20-HETE plays a role in myogenic activation of small arterioles of the cerebral and renal circulations (Harder D R et al., J Vasc Res 34: 237-243, 1997; Harder D R et al., Acta Physiol Scand 168: 543-549, 2000; and Ma Y H et al., Am J Physiol Regul Integr Comp Physiol 267: R579-R589, 1994). Frisbee et al. (Am J Physiol Heart Circ Physiol 280: H1066-H1074, 2001) and Kunert et al. (Microcirculation 8: 435-443, 2001) have demonstrated that 20-HETE contributes to the vasoconstrictor responses to elevations in transmural pressure and PO₂ in skeletal muscle resistance arterioles.

Recent studies have also suggested that norepinephrine and angiotensin II (ANG II) stimulate the synthesis and release of 20-HETE in vascular smooth muscle cells (Muthalif M M et al., J Biol Chem 271: 30149-30157, 1996) and that cytochrome P-450 inhibitors block activation of the MAPK system and the mitogenic effects of norepinephrine and ANG II on cultured vascular smooth muscle (VSM) cells. There is evidence that 20-HETE serves as a second messenger for the vasoconstrictor actions of ANG II (Alonso-Garcia M et al., Am J Physiol Regul Integr Comp Physiol 283: R60-R68, 2002) and the local renin-angiotensin system plays a critical role in angiogenesis induced by electrical stimulation (Amaral S L et al., Microcirculation 8: 57-67, 2001). However, the role of 20-HETE in mediating the angiogenic effects of Ang II following electrictical stimulation of muscle is not clear.

20-HETE has also been implicated to play a role in promoting the growth of various types of normal cells grown in culture. For instance, 20-HETE increases thymidine incorporation in VSM cells (Muthalif et al., Hypertension 36: 604-609, 2000; and Uddin et al., Hypertension 31: 242-247, 1998) and proximal tubule renal epithelial cells (Lin et al., Am. J. Physiol. 269: F806-F816, 1995). In both of these cell types in vitro the mitogenic effects of EGF were associated with an increase in the production of 20-HETE (Muthalif M M et al., Proc. Natl. Acad. Sci. USA 1998, 95:12701-12706; and Lin F et al., Am J Physiol 1995, 269:F806-16). Blockade of the formation of 20-HETE attenuated the growth response to serum, norepinephrine, and EGF (Lin F et al., Am J Physiol 1995, 269:F806-16; Roman R J, Physiol Rev 2002, 82:131-85; Sacerdoti D et al., Prostaglandins Other Lipid Mediat 2003, 72:51-71; Zhao X and Imig J D, Curr Drug Metab 2003, 4:73-84). Several signal transduction pathways may be involved in the stimulation of cell growth by 20-HETE in various cell types (Muthalif M M et al., Proc. Natl. Acad. Sci. USA 1998, 95:12701-12706; Uddin M R et al., Hypertension 31: 242-247, 1998; Harder D R et al., J Vasc Res 34: 237-243, 1997; Lange et al., J Biol Chem 272: 27345-27352, 1997; Lin et al., Am J Physiol Renal Physiol 269: F806-F816, 1995; and Sun et al., Hypertension 33: 414-418, 1999). Muthalif et al. reported that activation of MAPK by NE and angiotensin II in vascular smooth muscle cells is dependent on the formation of 20-HETE, which is generated following stimulation of cPLA₂ by calcium/calmodulin-dependent protein kinase II. Activation of the Ras/MAPK pathway by 20-HETE amplifies cPLA₂ activity and additional release of arachidonic acid by a positive feedback mechanism. Muthalif et al. proposed that this mechanism may play a role in the regulation of other cellular signaling molecules involved in cell proliferation and growth (Muthalif M M et al., Proc. Natl. Acad. Sci. USA 1998, 95:12701-12706).

Although the production of arachidonic acid metabolites is altered by growth factors that stimulate angiogenesis and there is evidence that 20-HETE may play a role in promoting the growth of some types of normal cells in vitro, prior to the work described in this application there is no evidence that 20-HETE is directly involved in angiogenesis in vivo and there is no evidence that 20-HETE plays a role in the proliferation of cancer cells and the growth of cancerous tumors.

### BRIEF SUMMARY OF THE INVENTION

In one aspect, the present invention relates to a method for reducing angiogenesis in a tissue of a human or non-human mammal by administering to the human or non-human mammal a 20-HETE synthesis inhibitor or 20-HETE antagonist in an amount sufficient to reduce angiogenesis in the tissue.

In another aspect, the present invention relates to a method for inducing and promoting angiogenesis in a tissue of a human or non-human mammal by sufficiently increasing 20-HETE activity in the tissue so that angiogenesis is induced and promoted.

In still another aspect, the present invention relates to a method for inhibiting cancer or tumor cell proliferation by exposing cancer or tumor cells to an agent selected from a 20-HETE synthesis inhibitor or a 20-HETE antagonist in an amount sufficient to inhibit proliferation of the cancer or tumor cells.

### BRIEF DESCRIPTION OF THE SEVERAL VIEWS OF THE DRAWINGS

FIG. 1 shows a representative reverse-phase HPLC chromatogram illustrating the separation of fluorescently labeled 20-hydroxyeicosatetraenoic acid (20-HETE) in samples from the tibialis anterior (TA) muscle. WIT-002, 20-5(Z), 14(Z)-hydroxyeicosadienoic acid, was used as an internal standard.

FIG. 2 shows the effects of treatment with a selective cytochrome P-450A (CYP4A) inhibitor [N-hydroxy-N′-(4-butyl-2-methylphenol)-formamidine (HET0016)] on 20-HETE levels in the urine of rats. Values are means±SE of 5 rats treated with vehicle (lecithin) and 5 rats treated with HET0016. *P<0.05 vs. lecithin.

FIG. 3 shows the effects of treatment with a selective CYP4A inhibitor (HET0016) on 20-HETE formation in the muscle of rats after 7 days of the stimulation protocol. PBS, phosphate-buffered saline. Values are means±SE of 5 rats treated with lecithin and 5 rats treated with HET0016. *P<0.05 vs. the unstimulated side.

FIG. 4 shows changes in vessel density of the extensor digitorum longus (EDL) and TA muscles in control rats (n=4), those treated with a selective CYP4A inhibitor (HET0016, 2 mg kg⁻¹ day⁻¹ in lecithin, n=4), and those treated with a nonselective CYP4A inhibitor [1-aminobenzotriazole (ABT), 50 mg kg⁻¹ day⁻¹ in PBS, n=4] after 7 days of the electrical stimulation protocol. Values are means±SE. Significance: *P<0.05 vs. the unstimulated side.

FIG. 5 shows the expression of vascular VEGF in TA muscle unstimulated (U) or electrically stimulated (S) from rats treated with lecithin, HET0016 (2 mg kg⁻¹ day⁻¹ in lecithin), and ABT (50 mg kg⁻¹ day⁻¹ in PBS, n=4) after 7 days of the electrical stimulation protocol. For each sample, 50 μg of total protein were loaded. C₆ tumor cells were used as a positive control as these cells avidly express VEGF. Quantitative densitometry of VEGF protein in the control group (n=5) and those treated with the selective CYP4A inhibitor HET0016 (n=7) after 7 days of the electrical stimulation protocol is shown. Values are means±SE. *P<0.05 vs. the unstimulated side.

FIG. 6 shows the effects of treatment with VEGF-neutralizing antibody (VEGF Ab; 0.6 mg/100 g body weight, ip in PBS) on 20-HETE formation in the muscle of rats after 7 days of the stimulation protocol. Values are means±SE of 5 rats treated with PBS (control) and 4 rats treated with VEGF Ab. Values are expressed as a percentage of VEGF expression seen in the C₆ tumor cell standard. *P<0.05 vs. the unstimulated side.

FIG. 7 shows the effects of HET0016 on the proliferative response of VEGF in cultured human vascular endothelial cells (HUVECs). HUVECs were incubated with 250 ng/ml VEGF alone or in the presence of 10 μM HET0016 and cell proliferation was assayed 24 hours later. HET0016 abolished the proliferative response to VEGF (n=3, each by triplicate), but did not alter basal proliferation rate of HUVECs (not shown). *p<0.05, control vs VEGF; p<0.05, VEGF vs VEGF+HET0016.

FIGS. 8A and 8B show the effects of HET0016 on the angiogenic response elicited by VEGF in vivo. Changes in neovascularization were assayed using the rat cornea pocket angiogenesis assay. Pellets containing VEGF alone (250 ng/pellet) or VEGF and HET0016 (20 μg) were implanted into the stroma of the cornea of rats. The rats were sacrificed 7 days later and neovascularization visualized using India ink. Total vessel length, a quantitative estimation of the angiogenic response, was measured by tracing the vessels and using image analysis software to obtain a numerical value of the length of all the vessels in the field. FIG. 8A shows representative cornea flat mounts in the region of the pellet implant. FIG. 8B shows total vessel length as a mean value±SEM for all experiments. (p<0.001, VEGF vs VEGF+HET0016).

FIGS. 9A and 9B show the effects of HET0016 on the angiogenic response elicited by bFGF in vivo. Pellets containing bFGF alone (250 ng/pellet) or bFGF and HET0016 (20 μg) were implanted into the stroma of the rat cornea. FIG. 9A shows representative cornea flat mounts. FIG. 9B shows changes in the angiogenic response as in FIG. 8. (n=6; p<0.001, bFGF vs bFGF+HET0016).

FIGS. 10A and 10B show the effects of HET0016 on the angiogenic responses elicited by EGF in vivo. Pellets containing EGF alone (250 ng/pellet) or EGF and HET0016 (20 μg) were implanted into the stroma of the rat cornea. FIG. 10A shows representative cornea flat mounts, and FIG. 10B shows changes in the angiogenic response (n=7 p<0.001, EGF vs EGF+HET0016).

FIGS. 11A and 11B show the effects of a chemically and mechanistically dissimilar inhibitor of the formation of 20-HETE, dibromododecenyl methylsulfonimide (DDMS, also called N-methylsulfonyl-12,12-dibromododecyl-11-enamide), on the angiogenic response to VEGF in vivo. Pellets containing VEGF alone (250 ng/pellet) or VEGF and DDMS (10 μg) were implanted into the stroma of the rat cornea. FIG. 11A shows a representative example and FIG. 11B shows the difference in the angiogenic response. (n=6; p<0.001, VEGF vs VEGF+DDMS).

FIG. 12 shows the effects of 20-hydroxyeicosa-6(Z),15(Z)-dienoic acid (WIT003), a more stable analog of 20-HETE with potent agonist activity, on the proliferation of cultured HUVECs. HUVECs were incubated with either 40 μM palmitic acid (inactive fatty acid control) or ethanol alone (vehicle control). There was no difference and therefore the control data from both of these groups were combined. In the experimental group, cells were incubated with 1 μM WIT003 for 48 hours and proliferation assayed. WIT003 increased proliferation in HUVECs (n=3, each by triplicate; *p<0.05 and #p<0.01, controls vs WIT003).

FIGS. 13A and 13B show the angiogenic effects of the 20-HETE analog, WIT003, in the rat cornea pocket assay in vivo. Pellets containing 20 μg WIT003 were implanted into the stroma of the cornea of rats. The rats were sacrificed 7 days later and neovascularization measured. FIG. 13A shows representative flat mounts. FIG. 13B shows the angiogenic response to WIT003 (n=6; p<0.01, controls vs WIT003).

FIGS. 14A and 14B show the effects of HET0016 on the angiogenic response of U251 cancer cells in vivo. Spheroids of the human glioblastoma cancer cell line U251 were generated by seeding single-cell suspensions at low densities over a layer of 0.8% agar. A total of 5-8 spheroids, approximately 200 μm each, were inserted into a corneal pocket carved in both eyes. Pellets containing either 20 μg HET0016 or vehicle (ethanol) were placed adjacent to the spheroids. Rats were sacrificed 2 weeks after implantation of the cancer cells and neovascularization responses measured. FIG. 14A shows the corneas in the region of the spheroid/pellet implant for all rats in this series. FIG. 14B shows changes in angiogenic response (n=8; p<0.01, control spheroids vs spheroids+HET0016).

FIG. 15 shows the effects of an inhibitor of the synthesis of 20-HETE, HET0016, on the growth pattern and cell cycle profile of human U251 glioblastoma cancer cells grown in vitro. Panel A: equal numbers of U251 cells (0.75×10⁴) were plated and serum starved for 1 day prior to the exposure of various concentrations of HET0016, and cell counting was performed every 24 h; Panel B: [³H]thymidine incorporation into DNA was assessed in control cultures and cultures treated with 10 μM HET0016. [³H]thymidine incorporation data were calculated as d.p.m./10³ cells and then normalized to the EtOH controls; Panel C: U251 cells were plated and treated with 10 μM HET0016 in EtOH or EtOH alone (control). Cells were stained with propidium iodide (a marker of cell proliferation), and their cell cycle distribution was determined analyzing total DNA content of the stained cells by FACS. Percentage of cells in various stages of the cell cycle is indicated in each figure. Mean±SD of three to four separate experiments performed in triplicate are presented in each panel. Panel C shows a representative experiment of three separate experiments. Arrow indicates the Time 0 when HET0016 was added to cultures.

FIG. 16 shows that HET0016 inhibits the effects of EGF to stimulate the proliferation and growth U251 cancer cells in culture. U251 cells were serum starved and then treated with 200 ng/ml EGF, 10 μM HET0016, or both. Cell proliferation was evaluated 48 hr later.

FIG. 17 shows a comparison of the effects of HET0016 on the growth of HUVECs, primary keratinocytes, and U251. HUVECs, primary human keratinocytes, and human U251 glioblastoma cancer cells were seeded onto 96-well plates and treated with HET0016 for 48 hrs. HET0016 had no effect on the proliferation of normal HUVECs or keratinocytes, while it inhibited the proliferation of U251 cancer cells by about 50%. Mean values±SD from three separate experiments, each performed in triplicate, are presented. *** indicates p<0.001 from the respective control values.

FIG. 18 shows the effects of DDMS on the proliferation of human U251 glioblastoma cells grown in culture. DDMS, a second CYP4A and 20-HETE synthesis inhibitor that is chemically and mechanistically quite different from HET0016, was used to treat the U251 cells. DDMS inhibited the proliferation of U251 cells in a concentration dependent manner. Mean±SD from three separate experiments, each performed in triplicate, are presented.

FIG. 19 shows the effects of WIT003, a stable 20-HETE analog with agonist properties, on the proliferation of human U251 glioblastoma cancer cells in vitro. U251 cultures were serum starved for 1 day before the addition of 0.1 μM and 1 μM of WIT003 or different concentrations of EGF which is known to produce near maximal stimulation of the proliferation of these cells for comparison. The results indicate that 1 μM WIT003 increases U251 growth as much as 200 ng/ml EGF. Cell numbers were counted 48 h later and normalized against values seen in control cultures treated with vehicle (EtOH) alone. Mean values±SD from three separate experiments, each performed in triplicate, are presented. *p<0.05; **p<0.01; ***p<0.001.

FIG. 20 shows that addition of the 20-HETE agonist WIT003 can rescue U251 cancer cells from the anti-proliferative effects of the 20-HETE synthesis inhibitor HET0016. The cultures were serum staved and treated with either 10 μM HET0016 alone or in combination with 1 μM WIT003. Cell proliferation was assessed by cell counting 48 h after treatment. Mean values±SD from three separate experiments, each performed in triplicate are presented.

FIG. 21 shows effects of HET0016 on the proliferation of 9L gliosarcoma cells in vitro. Panel A: equal numbers of 9L cells (0.75×10⁴) were plated and serum starved for 1 day prior to the exposure of various concentrations of HET0016 and cell counting was performed every 24 hr; Panel B: [³H]thymidine incorporation into DNA was assessed in cultures treated with 10 μM HET0016. [³H]thymidine incorporation data were calculated as d.p.m./10³ cells and then normalized to the EtOH controls. Data in panels A-B are Mean±SE from three separate experiments performed in triplicate.

FIG. 22 shows effects of DDMS on the proliferation of 9L gliosarcoma cells in vitro. Equal numbers of 9L cells (0.75×10⁴) were plated and serum starved for 1 day prior to the exposure of various concentrations of DDMS or vehicle and cell counting was performed 24 and 48 hrs later. Mean±SE of three separate experiments performed in triplicate are presented.

FIG. 23 shows effects of HET0016 on EGF-timulated 9L proliferation. 9L cultures were serum starved and then treated with 200 ng/ml EGF alone or EGF and 10 μM HET0016. Cell proliferation was evaluated at 24 and 48 hrs later.

FIG. 24 shows effects of a 20-HETE agonist WIT003 on the anti-proliferative effects of HET0016 of 9L gliosarcoma cells grown in vitro. Panel A: Cultures were serum staved and treated with either 0.1 μM or 1 μM WIT003. Cell numbers were assessed 48 hr later. Data are presented as % of control. Panel B: 9L cells were treated with either 10 μM HET0016 alone or in combination with 1 μM WIT003. Cell proliferation was assessed by cell counting 24 and 48 hrs after treatment. Data shown as % of inhibition. Mean±SE of three separate experiments performed in triplicate are presented.

FIG. 25 shows effects of HET0016 on the growth of 9L gliosarcoma tumors in vivo. 9L cells (1×10⁴) were injected into brains of rats. After 2 days for the tumors to become established the rats were treated with lecithin (vehicle) or HET0016 (10 mg/kg/day) for 15 days. Panel A: Brain tissue from control rat injected with lecithin (vehicle) is shown. Panel B: Brain tissue from HET0016 treated rat after 15 days of treatment is shown. Pictures shown are representative images seen in 5 control and 5 HET0016 treated animals.

FIG. 26 shows effects of chronic treatment of HET0016 on the growth of 9L tumors in vivo. Panel A presents HE sections through the midpoint of the tumors in rats treated with vehicle or HET0016. Panel B presents a comparison of the volume of the tumors in control and HET0016 rats measured in serial sections using an AIS Image Analysis System software. Mean±SE from 5 rats per group are presented.

### DETAILED DESCRIPTION OF THE INVENTION

It is disclosed here that angiogenesis can be regulated by modulating the activity of 20-HETE. It is further disclosed that cancer and tumor cell proliferation can be stimulated by 20-HETE and its agonists and inhibited by 20-HETE synthesis inhibitors and antagonists.

Using rat skeleton muscle and rat cornea as examples, the inventors have demonstrated that blocking 20-HETE synthesis with at least three different chemically and mechanistically distinct 20-HETE synthesis inhibitors reduced blood vessel development induced by various growth factors. Consistent with this observation, the inventors also demonstrated that administration of a 20-HETE agonist could mimic the effect of the growth factors to induce new blood vessel development. These discoveries provide new strategies for blocking angiogenesis to treat or prevent diseases and conditions associated with abnormal, excessive blood vessel development and new strategies for inducing and promoting angiogenesis to treat or prevent diseases and conditions associated with insufficient blood vessel development or blood vessel regression.

Using human glioma and rat gliosarcoma cancer cells as examples, the inventors have shown that chemically and mechanistically different types of 20-HETE synthesis inhibitors inhibit cancer cell proliferation both in vitro and in vivo and this inhibition could be reversed by a 20-HETE agonist. The inventors further found that 20-HETE synthesis inhibitors did not affect the basal proliferation of normal cells. However, the 20-HETE inhibitors blocked abnormal proliferation normal human vascular endothelial cells after the growth of these cells was abnormally stimulated using various growth factors (EGF, bFGF, and VEGF). These discoveries provide new strategies for cancer treatment (including adjunct therapies) and prevention.

The activity and synthesis of 20-HETE are well conserved among mammals. For example, enzymes of the CYP4A and CYP4F families are expressed and 20-HETE is produced by white blood cells and in blood vessels in all mammalian species studied to date (Roman R J., Physiol. Rev. 82:131-85, 2002). Therefore, the observations shown in the examples below using rats, rat cells, and human cells apply to all mammals such as humans, dogs, rats, mice, and rabbits.

In one aspect, the present invention relates to a method for reducing angiogenesis in a tissue of a human or non-human mammal by sufficiently inhibiting 20-HETE activity in the tissue so that angiogenesis is reduced.

In one embodiment, the method of the present invention is used to reduce angiogenesis induced by a growth factor. A skilled artisan is familiar with such growth factors. Examples include but are not limited to tyrosine kinase dependent growth factors such as VEGF, bFGF, EGF, Insulin, Insulin-like growth factor (IGF-1), and PDGF and G protein coupled receptors such as adrenergic, cholinergic, oxytocin, endothelin, angiotensin, bradykinin, histamine, thrombin, and many others.

In another embodiment, the method of the present invention is used to reduce the angiogenesis induced by the secretion of growth factors by tumor or cancer cells. In still another embodiment, the method of the present invention is used to reduce angiogenesis in a non-muscle tissue such as a non-muscle tissue in the eye (e.g., following exposure of newborns to high oxygens, following injury and inflammation, and in diabetes). In still another embodiment, the method of the present invention is used to reduce angiogenesis in a non-muscle tissue in inflammatory conditions such as asthma, rheumatoid arthritis, osteoarthritis, skin infections and injury, and pulmonary fibrosis.

One suitable way to inhibit 20-HETE activity in a tissue of a human or non-human mammal is to administer a 20-HETE synthesis inhibitor to the human or non-human mammal in an amount sufficient to reduce angiogenesis in the tissue. By “20-HETE synthesis inhibitor,” we mean an inhibitor of an enzyme that is involved in converting arachidonic acid to 20-HETE. Such enzymes are known and include those of the CYP4A and CYP4F families such as CYP4A11, CYP4F2, and CYP4F3 (Christmas P et al., J. Biol. Chem., 276: 38166-38172, 2001).

Many classes of 20-HETE synthesis inhibitors are known in the art and they can all be used in the methods of the present invention. These inhibitors include those disclosed in U.S. Ser. No. 20040110830; WO0236108; WO0132164; Nakamura T et al., Bioorg Med Chem. 12:6209-6219, 2004; Nakamura T et al., Bioorg Med Chem Lett. 14:5305-5308, 2004; Nakamura T et al., Bioorg Med Chem Lett. 14:333-336, 2004; Nakamura T et al., J Med Chem. 46:5416-5427, 2003; Sato M et al., Bioorg Med Chem Lett. 11:2993-2995, 2001; Miyata N et al., Br J Pharmacol. 133:325-329, 2001; Xu F et al., J Pharmacol Exp Ther. 308:887-895, 2004; Xu F et al., Am J Physiol Regul Integr Comp Physiol 28:R710-720, 2002; Roman R J., Physiol Rev. 82:131-185, 2002, all of which are herein incorporated by reference in their entirety.

Examples of these inhibitors include N-hydroxy-N-(4-butyl-2-methylphenyl)-formamidine (HET0016), N-(3-Chloro-4-morpholin-4-yl)phenyl-N′-hydroxyimidoformamide (TS-011), dibromododecenyl methylsulfonimide (DDMS), 1-aminobenzotriazole (ABT, available from Sigma Chemical Corp., St. Louis, Mo.), 17-Octadecynoic acid (17-ODYA), miconazole (available from Sigma Chemical Corp., St. Louis, Mo.), ketoconazole, fluconazole, and 10 undecynyl sulfate (10-SUYS). HET0016, TS-011, and DDMS are 20-HETE specific inhibitors and 17-ODYA, 1-ABT, and miconazole are less specific inhibitors (WO0236108). HET0016, 1-ABT, and 17-ODYA have been shown to be able to reduce 20-HETE levels in vivo (WO0236108; Dos Santos E A et al., Am J Physiol Regul Integr Comp Physiol. 287:R58-68, 2004; Hoagland K M et al., Hypertension 42:669-673, 2003; Cambj-Sapunar L et al., Stroke 34:1269-1275, 2003; and Hoagland K M et al., Hypertension 41:697-702, 2003). A method for synthesizing HET0016 is disclosed in WO0132164. The synthesis of a large number of analogs of HET0016 with similar properties to inhibit the synthesis of 20-HETE have also been described (Nakamura T et al., Bioorg Med Chem. 12:6209-6219, 2004; Nakamura T et al., Bioorg Med Chem Lett. 14:5305-5308, 2004; Nakamura T et al., Bioorg Med Chem Lett. 14:333-336, 2004; Nakamura T et al., J Med Chem. 46:5416-5427, 2003; and Sato M et al., Bioorg Med Chem Lett. 11:2993-2995, 2001). 17-ODYA, ABT, and miconazole are available from Sigma Chemical Corp., St. Louis, Mo. Preferred inhibitors for the purpose of the present invention include HET0016, TS-011, and DDMS.

U.S. Ser. No. 20040110830 discloses hydroxyformamidine derivatives that can inhibit 20-HETE synthesis from arachidonic acid and all these derivatives can be used in the present invention.

Antibodies (monoclonal or polyclonal) against a 20-HETE synthesizing enzyme can also be used as 20-HETE synthesis inhibitors as it has been shown in general that an antibody can block the function of a target protein when administered into the body of an animal (Dahly, A. J., FASEB J. 14:A133, 2000; Dahly, A. J., J. Am. Soc. Nephrology 11:332A, 2000). The DNA and protein amino acid sequences of all known members of the CYP4A and CYP4F families are published and available. A skilled artisan can thus make antibodies including humanized antibodies to a 20-HETE synthesis enzyme. For example, antibodies against CYP4A1 and CYP4A10 have been made and shown to be capable of inhibiting the enzyme activity of CYP4A1 and CYP4A10 (Amet, Y. et al., Biochem Pharmacol. 54(8): 947-952, 1997; Amet, Y. et al., Biochem. Pharmacol. 53(6): 765-771, 1997; Amet, Y. et al., Alcohol Clin. Exp. Res. 22(2): 455-462, 1998). Certain such antibodies are also commercially available (e.g., anti-CYP4A1 is available from Gentest Corp., Woburn, Mass.).

Another suitable way to inhibit the 20-HETE activity in a tissue of a human or non-human mammal is to administer a 20-HETE antagonist to the human or non-human mammal in an amount sufficient to reduce angiogenesis in the tissue. All known 20-HETE antagonists can be used. These include those disclosed in U.S. Pat. No. 6,395,781; Yu M et al., Eur J Pharmacol. 486:297-306, 2004; Yu M et al., Bioorg Med Chem. 11:2803-2821, 2003; and Alonso-Galicia M et al., Am J Physiol. 277:F790-796, 1999, all of which are herein incorporated by reference in their entirety. Examples include 19 hydroxynonadecanoic acid, 20 hydroxyeicosa-5(Z),14(Z), dienoic acid and N-methylsulfonyl-20-hydroxyeicosa-5(Z),14(Z)-dienamide.

Diseases and conditions that are associated with abnormal, excessive blood vessel development can be treated or prevented by the method of the present invention. By treating a disease, we mean reducing the severity of a disease after the development of the disease or making the symptoms of a disease disappear. By preventing a disease, we mean preventing the development of a disease or reducing the severity of the disease at its onset. Examples of the diseases and conditions that can be treated or prevented include but are not limited to cancer (e.g., brain cancer and other solid tissue tumors), vascularization of the cornea in newborns placed in high oxygen incubators, and vascularization of the eye, skin, and other organs following injury or infection. In addition to vascularization as a result of injury or infection, other eye diseases such as neovascular eye diseases caused by uncontrolled angiogenesis can also be treated or prevented. In this instance, it is noted that pathological angiogenesis from retinal and choroidal circulations is a serious consequence of many eye diseases. Retinal neovascularization occurs in diabetic retinopathy, sickle cell retinopathy, retinal vein occlusion, and retinopathy of prematurity (ROP). Occlusion of the central retinal vein, or one of its branches, can lead to rapid diminution of vision with later sequelae of retinal neovascularization. The vascular supply to the optic nerve, derived from the choroidal system, may be interrupted in anterior ischemic optic neuropathy. New blood vessels arising from choroidal capillaries lead to choroidal neovascularization which occurs in age-related macular degeneration and several macular diseases.

Inappropriate angiogenesis is also involved in deleterious remodeling in atherosclerosis and restenosis, idiopathic pulmonary fibrosis, acute adult respiratory distress syndrome, and asthma. In addition, angiogenesis has been associated with arthritic diseases such as rheumatoid arthritis. All of these diseases can be treated or prevented with the method of the present invention. In addition to the diseases and conditions described above, other diseases and conditions that can be treated or prevented include but are not limited to those listed in Table 1 (Carmeliet P, Nature Medicine 9:653-660, 2003; and Carmeliet P, J. Intern. Med. 255:538-561, 2004, both are incorporated by reference in their entirety). Other information regarding the diseases can be found in Storgard C M, et al., J Clin Invest. 103:47-54 (1999) and Greene A S and Amaral S L, Curr Hypertens Rep. 4:56-62 (2002), all of which are herein incorporated by reference in their entirety.

TABLE 1
Diseases characterized or caused by abnormal or excessive angiogenesis

| Organ | Diseases in mice or humans |
|---|---|
| Numerous organs | Cancer (activation of oncogenes; loss of tumor suppressors); |
| | infectious diseased (pathogens express angiogenic genes, induce |
| | angiogenic programs or transform ECs); autoimmune disorders |
| | (activation of mast cells and other leukocytes) |
| Blood vessels | Vascular malformations (Tie-2 mutation); DiGeorge syndrome |
| | (low VEGF and neuropilin-1 expression); HHT (mutations of |
| | endoglin or ALK-1); cavernous hemangioma (loss of Cx37 and |
| | Cx40); atherosclerosis; transplant arteriopathy |
| Adipose tissue | Obesity (angiogenesis induced by fatty diet; weight loss by |
| | angiogenesis inhibitors) |
| Skin | Psoriasis, warts, allergic dermatitis, scar keloids, pyogenic |
| | granulomas, blistering disease, Kaposi sarcoma in AIDS patients |
| Eye | Persistent hyperplastic vitreous syndrome (loss of Ang-2) or |
| | VEGF); diabetic retinopathy; retinopathy of prematurity; |
| | choroidal neovascularization (TIMP-3 mutation) |
| Lung | Primary pulmonary hypertension (germline BMPR-2 mutation; |
| | somatic EC mutations); asthma; nasal polyps |
| Intestines | Inflammatory bowel and periodontal disease, ascites, peritoneal |
| | adhesions |
| Reproductive system | Endometriosis, uterine bleeding, ovarian cysts, ovarian |
| | hyperstimulation |
| Bone, joints | Arthritis, synovitis, osteomyelitis, osteophyte formation |

In another aspect, the present invention relates to a method for reducing angiogenesis in a tissue of a human or non-human mammal by administering HET0016 or DDMS to the mammal in an amount sufficient to reduce angiogenesis in the tissue.

In another aspect, the present invention relates to a method for reducing angiogenesis in a tissue of a human or non-human mammal by administering TS-011 to the mammal in an amount sufficient to reduce angiogenesis in the tissue.

In another aspect, the present invention relates to a method for inducing and promoting angiogenesis in a tissue of a human or non-human mammal by sufficiently increasing 20-HETE activity in the tissue so that angiogenesis is induced and promoted. In one embodiment, the method is used to induce and promote angiogenesis in a non-muscle tissue.

One suitable way to increase 20-HETE activity in a tissue of a human or non-human mammal is to administer 20-HETE or one of its agonists to the human or non-human mammal in an amount sufficient to induce and promote angiogenesis in the tissue. All known 20-HETE agonists can be used. These include those disclosed in U.S. Pat. No. 6,395,781; Yu M et al., Eur J Pharmacol. 486:297-306,2004; Yu M et al., Bioorg Med Chem. 11:2803-2821, 2003; and Alonso-Galicia M et al., Am J Physiol. 277:F790-796, 1999. Examples include 20 hydroxyeicosanoic acid, 20 hydroxyeicosa-6(Z),15(Z)-dienoic acid (WIT003), and N-methylsulfonyl-20-hydroxyeicosa-6(Z),15(Z)-dienamide.

Diseases and conditions associated with insufficient angiogenesis or vessel regression can be prevented or treated by the method provided here. For example, peripheral vascular diseases associated with diabetes and ischemic heart disease can be prevented or treated. Therapeutic angiogenesis should help to reduce the need for limb amputation in patients with peripheral vascular disease, and in the case of revascularizing the heart, this therapy should augment survival following heart attack and should augment or even replace bypass surgery. Similarly, administration of 20-HETE or its agonists to increase angiogenesis should mitigate cell death and neurological deficits following ischemic stroke and in conditions associated with reduced vascularization of areas of the brain (e.g., Alzhiemer disease). Other diseases and conditions that can be prevented or treated include but are not limited to those listed in Table 2 below (Carmeliet P, J. Intern. Med. 255:538-561, 2004).

TABLE 2
Disease characterized or caused by insufficient angiogenesis or vessel regression

| Organ | Disease in mice or humans | Angiogenic mechanism |
|---|---|---|
| Nervous system | Alzheimer disease | Vasoconstriction, microvascular |
| | | degeneration and cerebral angiopathy due |
| | | to EC toxicity by amyloid-β |
| | Amyotrophic lateral | Impaired perfusion and neuroprotection, |
| | sclerosis; diabetic | causing motoneuron or axon degeneration |
| | neuropathy | due to insufficient VEGF production |
| | Stroke | Correlation of survival with angiogenesis in |
| | | brain; stroke due to arteriopathy (Notch-3 |
| | | mutations) |
| Blood vessels | Atherosclerosis | Characterized by impaired collateral vessel |
| | | development |
| | Hypertension | Microvessel rarefaction due to impaired |
| | | vasodilation or angiogenesis |
| | Diabetes | Characterized by impaired collateral growth |
| | | and angiogenesis in ischemic limbs, but |
| | | enhanced retinal neovascularization |
| | | secondary to pericyte dropout |
| | Restenosis | Impaired re-endothelialization after arterial |
| | | injury at old age |
| Gastrointestinal | Gastric or oral ulcerations | Delayed healing due to production of |
| | | angiogenesis inhibitors by pathogens |
| | Crohn disease | Characterized by mucosal ischemia |
| Skin | Hair loss | Retarded hair growth by angiogenesis |
| | | inhibitors |
| | Skin purpura, telangiectasia | Age-dependent reduction of vessel number |
| | and venous lake formation | and maturation (SMC dropout) due to EC |
| | | telomere shortening |
| Reproductive | Pre-eclampsia | EC dysfunction resulting in organ failure, |
| System | | thrombosis and hypertension due to |
| | | deprivation VEGF by soluble Flt-1 |
| | Menorrhagia | Fragility of SMC-poor vessels due to low |
| | (uterine bleeding) | Ang-1 production |
| Lung | Neonatal respiratory distress | Insufficient lung maturation and surfactant |
| | | production in premature mice due to |
| | | reduced HIF-2α and VEGF production |
| | Pulmonary fibrosis, | Alveolar EC apoptosis upon VEGF |
| | emphysema | inhibition |
| Kidney | Nephropathy | Age-related vessel loss due to TSP-1 |
| | | production |
| Bone | Osteoporosis, impaired bone | Impaired bone formation due to age |
| | fracture healing | dependent decline of VEGF-driven |
| | | angiogenesis; angiogenesis inhibitors |
| | | prevent fracture healing |

In another aspect, the present invention relates to a method for inducing and promoting angiogenesis in a tissue of a human or non-human mammal by administering 20 hydroxyeicosa-6(Z),15(Z)-dienoic acid to the mammal in an amount sufficient to induce and promote angiogenesis in the tissue.

In another aspect, the present invention relates to a method for inhibiting tumor or cancer cell proliferation by exposing tumor or cancer cells to an agent selected from a 20-HETE synthesis inhibitor or a 20-HETE antagonist in an amount sufficient to inhibit proliferation of the tumor or cancer cells. In one embodiment, the agent is administered to a human or non-human mammal having cancer or tumor to treat the cancer or tumor. In another embodiment, the agent is administered to prevent the development of cancer or tumor.

Cancer cells are known for their ability to produce autocrine growth factors that contribute to their abnormal growth. In the examples below, the inventors have demonstrated that 20-HETE plays a role in mediating the cellular mitogenic responses to growth factors. Without intending to be limited by theory, the inventors believe that 20-HETE synthesis inhibitors and 20-HETE antagonists inhibit cancer or tumor cell proliferation by inhibiting the signal transduction pathway of the growth factors. In addition, the growth of the above cancer or tumor cells also depend on the secretion of growth factors to stimulate angiogenesis and provide a blood supply to the tumor. The present disclosure demonstrates that inhibitors of the synthesis and actions of 20-HETE prevent growth factor-induced angiogenesis in at least 2 different model systems in vivo. It is therefore further theorized that this inhibition of tumor-induced angiogenesis by the 20-HETE synthesis inhibitors and antagonists also contributes to their anti-cancer activity in vivo. In a preferred embodiment, 20-HETE synthesis inhibitors and 20-HETE antagonists are employed to prevent or treat brain cancers of glial cell origin (glioma) and astrocyte origin (astrocytoma) as well as cancers of epithelial tissues (carcinomas) such as certain types of intestinal cancer, breast cancer (e.g., ductal breast cancer), skin cancer, lung cancer, stomach cancer, prostate cancer, thyroid cancer, liver cancer, pancreatic cancer, kidney cancer, colon cancer, and ovarian cancer. In a more preferred embodiment, glioma and carcinomas of breast cancer, prostate cancer, colon cancer, skin cancer, and pancreatic cancer are prevented or treated. Suitable and preferred 20-HETE synthesis inhibitors and 20-HETE antagonists are as described above.

In another aspect, the present invention relates to a method for inhibiting tumor or cancer cell proliferation by exposing tumor or cancer cells to HET0016 or DDMS in an amount sufficient to inhibit proliferation of the tumor or cancer cells. In one embodiment, HET0016 or DDMS is administered to a human or non-human mammal having cancer or tumor to treat the cancer or tumor. In another embodiment, HET0016 or DDMS is administered to prevent the development of cancer or tumor.

In another aspect, the present invention relates to a method for inhibiting tumor or cancer cell proliferation by exposing tumor or cancer cells to TS-011 in an amount sufficient to inhibit proliferation of the tumor or cancer cells. In one embodiment, TS-011 is administered to a human or non-human mammal having cancer or tumor to treat the cancer or tumor. In another embodiment, TS-011 is administered to prevent the development of cancer or tumor.

For a particular application of the present invention such as the prevention or treatment of a particular disease or condition, the optimal dosage of a particular 20-HETE synthesis inhibitor or a 20-HETE agonist or antagonist can be readily determined by a skilled artisan for a specific route of administration. The present invention is not limited by a specific route of administration. Suitable routes of administration include but are not limited to oral, intravenous, subcutaneous, intramuscular, and injection into a specific organ or tissue.

The invention will be more fully understood upon consideration of the following non-limiting examples.

### EXAMPLE 1

### Modulation of Skeletal Muscle Angiogenesis by 20-HETE

This example demonstrates that 20-hydroxyeicosatetraenoic acid (20-HETE) is important for angiogenesis induced by electrical stimulation in skeletal muscle. The tibialis anterior and extensor digitorum longus muscles of rats were stimulated for 7 days. Electrical stimulation significantly increased the 20-HETE formation and angiogenesis in the muscles, which was blocked by chronic treatment with N-hydroxy-N′-(4-butyl-2-methylphenol)formamidine (HET0016) or 1-aminobenzotriazole (ABT). Chronic treatment with either HET0016 or ABT did not block the increases in VEGF protein expression in both muscles. To analyze the role of VEGF on 20-HETE formation, additional rats were treated with VEGF-neutralizing antibody (VEGF Ab). VEGF Ab blocked the increases of 20-HETE formation induced by stimulation. These results place 20-HETE in the downstream signaling pathway for angiogenesis (downstream of VEGF).

Materials and Methods

Animal surgery: All protocols were approved by the Institutional Animal Care and Use Committee of the Medical College of Wisconsin. The rats were housed in the Animal Resource Center of the Medical College of Wisconsin and were given food and water ad libitum. Thirty-two male Sprague-Dawley rats, 7-8 wk old, were anesthetized with an intramuscular injection of a mixture of ketamine (100 mg/kg) and acepromazine (2 mg/kg). A subcutaneous incision was made over the thoracolumbar region, and a miniature battery-powered stimulator, which was previously designed and validated for chronic studies (Linderman J R et al., Microcirculation 7: 119-128, 2000), was implanted and secured in place. Another incision was made in the skin and fascia covering the lateral side of the knee joint (over the region of the common peroneal nerve) of the right hindlimb. A pair of electrodes was guided under the skin from the stimulator and secured to the muscles surrounding the knee in close proximity to the common peroneal nerve (Ma Y H et al., Am J Physiol Regul Integr Comp Physiol 267: R579-R589, 1994). The electrodes were locally secured into place using biocompatible acrylic cement (Loctite; Rocky Hill, Conn.) and distally with a fine suture (size 5-0, Ethicon; Somerville, N.J.). The skin over both incisions was sutured closed, and the rats were allowed to recover before the initiation of the stimulation period the following day.

Experimental protocols and tissue preparation: After a 24-h recovery period, the implanted stimulator was activated by momentary closure of the magnetic reed switch using a small hand-held magnet. The stimulator produced electrically induced muscle contractions in the lower leg muscles by stimulating the common peroneal nerve with square wave impulses of 0.3-ms duration, 10-Hz frequency, and 3-V potential (Linderman J R et al., Microcirculation 7: 119-128, 2000). Contractions of the extensor digitorum longus (EDL) and tibialis anterior (TA) muscles were automatically initiated at 9 AM each day and sustained for 8 h/day over a consecutive 7-day period. At the end of the stimulation period, the animals were euthanized by an overdose of pentobarbital sodium (100 mg/kg ip), and the EDL and TA muscles were harvested for analysis as previously described (Greene A S et al., Hypertension 15: 779-783, 1990; and Parmentier J H et al., Hypertension 37: 623-629, 2001).

The rats were divided in four groups. To evaluate the role of 20-HETE in contributing to the VEGF protein expression and skeletal muscle angiogenesis, nine rats in group 1 received two daily intraperitoneal injections of a potent and selective inhibitor of the CYP4A enzymes [N-hydroxy-N′-(4-butyl-2-methylphenol)formamidine (HET0016), Taisho Pharmaceutical (Miyata N et al., Br J Pharmacol 133: 925-929, 2001)] at a dose of 1 mg/kg each injection during the period of electrical stimulation. This dose was chosen based on our previous results (Kehl F et al., Am J Physiol Heart Circ Physiol 282: H1556-H1565, 2002). In that study, a dose of 10 mg/kg iv produced plasma concentrations that far exceeded (10 times higher) the effective inhibitory concentration of HET0016 in plasma for many hours.

To compare the effects of HET0016 with a more commonly used, but less specific inhibitor, four rats were treated with 1-aminobenzotriazole (ABT; group 2) at a dose of 50 mg·kg⁻¹·day⁻¹ ip during the period of electrical stimulation.

To determine the contribution of VEGF to the angiogenesis induced by electrical stimulation, six rats in group 3 were treated with 3 mg/kg ip injections of a monoclonal VEGF-neutralizing antibody (Texas Biotechnology; Houston, Tex.) during the period of electrical stimulation. The protocol for administration of VEGF-neutralizing antibody was modified from Zheng W et al. (Circ Res 85: 192-198, 1999), and this dose was based on our previous results (Amaral S L et al., Microcirculation 8: 57-67, 2001). After the stimulation period was started, the rats received intraperitoneal injections on days 3, 5, and 7 (0.6 mg/100 g).

In group 4, rats were treated with either the vehicle for HET0016, lecithin (n=9), or saline for VEGF antibody, PBS (n=4). Because there was no significant difference in the results obtained in rats treated with either vehicle, the results from those groups were pooled.

Measurement of urinary excretion of 20-HETE: On the last day of electrical stimulation, rats were placed in a metabolic cage that efficiently separates urine from food. Just before the start of the urine collection, food was withdrawn to avoid contamination of the urine sample, and the 24-h control and treated urine samples were collected into a glass bottle packed with ice. The concentration of 20-HETE in the urine samples was measured using a fluorescent HPLC assay, as previously described (Maier K G et al., Am J Physiol Heart Circ Physiol 279: H863-H871, 2000). After the addition of 25 ng of an internal standard [20-5(Z),14(Z)-hydroxyeicosadienoic acid (WIT-002), Taisho Pharmaceutical; Saitama, Japan] the samples were acidified to pH 4 with formic acid and extracted with 1 ml of ethyl acetate, and the organic phase was dried using argon gas. The samples were redissolved in 1 ml of 20% acetonitrile and loaded onto a Sep-Pak Vac column (Waters; Milford, Mass.). The column was washed twice with 1 ml of 30% acetonitrile, and the fraction containing HETEs and EETs was eluted with 400 μl of 90% acetonitrile. The samples were diluted in water, applied to a Sep-Pak Vac column, eluted with 500 μl of ethyl acetate, and then dried down. The lipid fraction was labeled with 20 μl of acetonitrile containing 36.4 mM 2-(2,3-napthalimino)ethyl trifluoromethanesulfonate. N,N-diisopropylethylamine (10 μl) was added to catalyze the reaction. Excess dye was removed using Sep-Pak Vac extraction (Maier K G et al., Am J Physiol Heart Circ Physiol 279: H863-H871, 2000), and the samples were dried under argon, resuspended in 100 μl of methanol, and analyzed by reverse-phase HPLC (Waters) using a fluorescence detector (model number L-7480; Hitachi, Naperville, Ill.). The amount of 20-HETE in the sample was determined by comparing the area of the 20-HETE peak with that of the internal standard.

Tissue harvest and morphological analysis of vessel density: The stimulated and contralateral muscles were removed, weighed, and rinsed in physiological salt solution. A 300-mg sample was taken from the rostral portion of the TA muscle and frozen in liquid nitrogen for Western blot (100 mg) and HPLC (200 mg) analysis for measurement of VEGF protein expression and 20-HETE formation, respectively. The remaining TA and EDL muscles were lightly fixed in a 0.25% formalin solution overnight. The muscles were sectioned via a manual microtome to a thickness of about 100 μm by securing the tendons and slicing parallel to the longitudinal orientation of the muscle fibers. From every animal, two slices of each EDL muscle and three slices of each TA muscle were made. The slices were than immersed for 2 h in a solution of 25 μg/ml rhodamine-labeled Griffonia simplicifolia I (GS-I) lectin (Sigma; St. Louis, Mo. (Greene A S et al., Hypertension 15: 779-783, 1990)). Immediately after this 2-h exposure to GS-I lectin, the muscles were rinsed in physiological solution. The rinsing procedure was repeated after 15 and 30 min, and the muscles were rinsed in physiological saline solution for 12 h (overnight, at 4° C.). On the next day, the slices were mounted on microscope slides with a water-soluble mounting medium consisting of toluene and acrylic resin (SP ACCU-MOUNT 280, Baxter Scientific).

The labeled sections were visualized using a video fluorescent microscope system (Olympus ULWD CD Plan, ×20 objective, 1.6 cm working distance and 0.4 numerical aperture) with epi-illumination, as previously described (Parmentier J H et al., Hypertension 37: 623-629, 2001). In the present study, 10-15 and 20-25 representative fields were selected for study from each EDL and TA muscle slice, respectively. Each field was converted to a digitized image (DT2801 Data Translation; Marlboro, Mass.) and stored as an 8-bit/pixel image file with a resolution of 512×512 pixels. Morphometric analysis of the scanned histochemical sections was done as previously described (Parmentier J H et al., Hypertension 37: 623-629, 2001). Vessel-grid intersections have been previously demonstrated to provide an accurate and quantitative estimate of vessel density (Parmentier J H et al., Hypertension 37: 623-629, 2001).

Western blot analysis to detect the presence of VEGF protein: The 100-mg TA muscle specimens were homogenized, and the protein was suspended in potassium buffer (10 mM). Five micrograms of protein (as determined by a protein assay kit, Bio-Rad; Hercules, Calif.) from the TA and a tumor cell line known to express VEGF at high levels (C₆, American Type Culture Collection, 107-CCL) were separated on a 12% denaturing polyacrylamide gel. The gels were transferred to a nitrocellulose membrane, which was blocked overnight in 5% nonfat dry milk diluted in Tris-buffered saline (50 mM Tris and 750 mM NaCl, pH 8) with 0.08% Tween 20 (Bio-Rad). The blots were then incubated with a polyclonal antibody to a peptide derived from the human VEGF sequence (1:1,000 dilution, clone G143-850, Pharmingen) for 2 h at room temperature. Washed blots were then incubated with goat anti-mouse secondary antibody at a dilution of 1:1,000 for 1 h at room temperature and then subjected to a SuperSignal West Dura chemiluminescence substrate (Pierce; Rockford, Ill.) detection system. Membranes were exposed to X-ray film (Fuji Medical; Stamford, Conn.) for 15 to 30 s and developed using a Kodak M35 X-Omat processor. For the quantitative VEGF analysis, film was always exposed for a period of time that ensured that all signals were within the linear range of the detection of the film. The VEGF band intensity was quantified using a morphometry imaging system (Metamorph, Universal Imaging; West Chester, Pa.), and values are expressed as a percentage of the C₆ tumor cell standard.

Muscle preparations for measurement of 20-HETE: The 100-200 mg of frozen TA muscle were homogenized in a solution containing 1 ml of acidified water and 50 μl of an internal standard, WIT-002, which was synthesized and kindly provided by Taisho Pharmaceutical. Ethyl acetate (3 ml, Fisher Scientific; Pittsburgh, Pa.) was added to the mixture and gently vortexed. The homogenized tissues were then centrifuged at 3,000 revolutions/min for 2 min. With the use of a glass Pasteur pipet, the top layer was removed and transferred to a sterile glass vial, and the samples were dried under nitrogen and stored at −80° C.

Labeling of samples and fluorescent detection of 20-HETE: Detection of 20-HETE was performed as previously described (Ma Y H et al., Am J Physiol Regul Integr Comp Physiol 267: R579-R589, 1994). Samples, which were extracted and dried under argon, were resuspended in 20 μl of acetonitrile containing 36.4 mM 2-(2,3-napthalimino)ethyl trifluoromethanesulfonate, and N,N-diisopropylethylamine (10 μl) was added as a catalyst. The sample was reacted for 30 min at room temperature, dried under argon, resuspended in 1 ml of 40% acetonitrile-water, and applied to a Sep-Pak Vac column. The column was washed with 6 ml of 50% acetonitrile-water solution to remove unreacted dye, eluted with 500 μl of ethyl acetate, dried under argon, and resuspended in 100 μl of the HPLC mobile phase [methanol-water-acetic acid, 82:18:0.1 (vol/vol)]. A 25-μl aliquot of the derivatized sample was separated on a 4.6×250-mm Symmetry C18 reverse-phase HPLC column (Waters) isocratically at a rate of 1.3 ml/min using methanol-water-acetic acid (82:18:0.1 (vol/vol)) as the mobile phase. Fluorescence intensity monitored using an in-line fluorescence detector (model number L-7480, Hitachi; Naperville, Ill.) at medium gain sensitivity. The amount of 20-HETE in the sample was determined by comparing the area of the 20-HETE peak with that of the internal standard (WIT-002).

Data analysis and statistics: For each muscle, the vessel counts of all the selected fields (10-15 scans×2 slices for each EDL muscle; 20-25 scans×3 slices for each TA muscle) were averaged to a single vessel density. Vessel density was expressed in terms of the mean number of vessel-grid intersections per microscope field (0.224 mm²). For each experimental group, the measured vessel density and 20-HETE formation of the stimulated muscle was compared with its unstimulated counterpart as well as with age-matched controls. All values are presented as means±SE. The significance of differences in values measured in the same animal was evaluated using a two-factor ANOVA (drug×stimulation) with repeated measures on one factor (stimulation). Significant differences were further investigated using a post hoc test (Tukey's).

Results

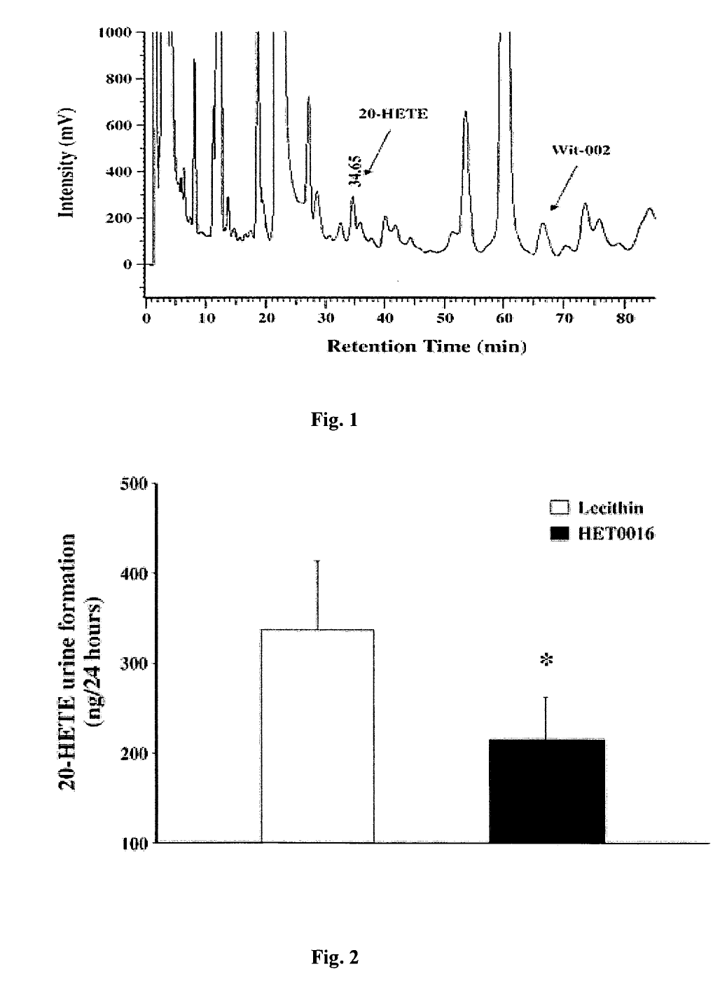

To evaluate the effect of the blockade of CYP4A enzymes, we measured the urinary 20-HETE excretions in rats treated with HET0016 for 7 days. A representative HPLC chromatogram illustrating the separation of 20-HETE is presented in FIG. 1. As shown in FIG. 1, there are other peaks very close to 20-HETE. On the basis of comigration of standards, we identified the preceding peak in the chromatogram as 19-HETE and the one after 20-HETE peak as 18-HETE. The next peak is 16-HETE, followed by 15-HETE. To analyze the area of each peak, we subtracted the shouldering peaks by using a deconvolution procedure (Hitachi software), as described in Materials and Methods.

As shown in FIG. 2, chronic treatment with HET0016 significantly reduced (by 36%) the 24-h urinary 20-HETE excretion compared with the control group, which was treated with lecithin (P<0.05).

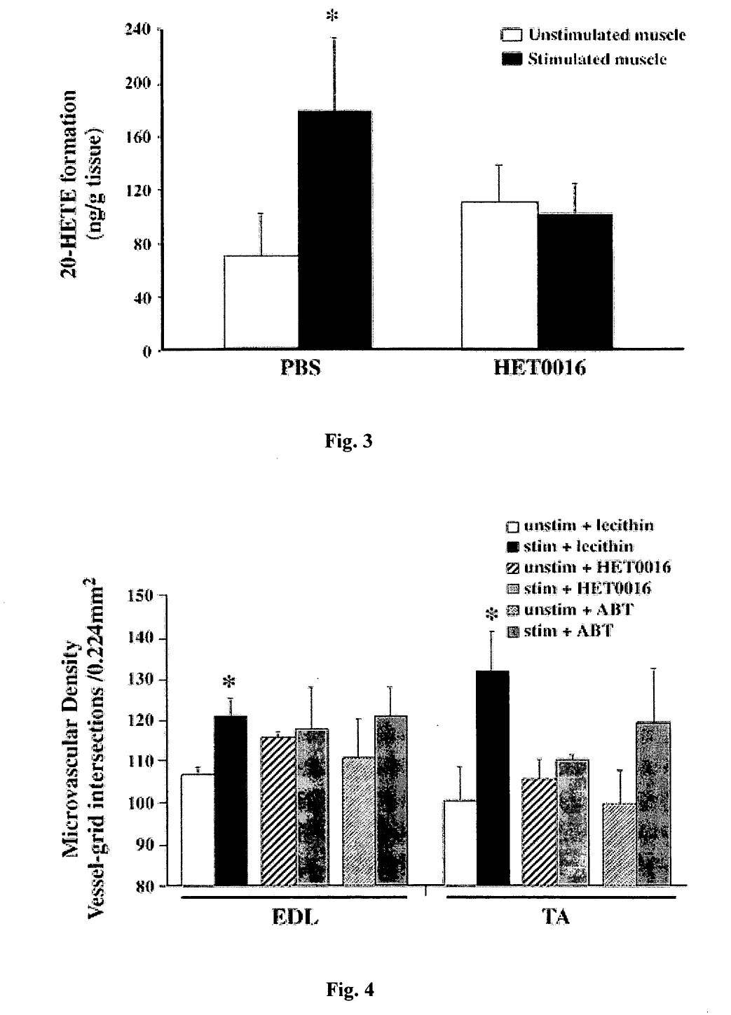

Seven days of electrical stimulation significantly increased the 20-HETE formation in skeletal muscle, as demonstrated in FIG. 3 (from 69.52±31.3 to 177.58±54.4 ng/g muscle for unstimulated and stimulated muscles, respectively, P<0.05). Treatment with HET0016 for 7 days did not change the basal formation of 20-HETE in skeletal muscle (110.26±28.36 and 69.52±31.3 ng/g muscle for treated and control, respectively, P>0.05; FIG. 3); however, chronic treatment with HET0016 completely blocked the increase of 20-HETE formation induced by electrical stimulation in skeletal muscle (from 110.26±28.3 to 102.1±22.3 ng/g muscle for the unstimulated and stimulated sides, respectively; FIG. 3).

Electrical stimulation, as has been shown previously, produced an increase in vessel density in the control group, which was treated with lecithin (from 107.0±1.6 to 121.0±4.5 and from 100.4±8.4 to 132.0±9.9 number of vessel intersections for the EDL and TA, respectively, P<0.05). As shown in FIG. 4, chronic treatment with HET0016 completely blocked the increase in vessel density induced by 7 days of electrical stimulation in skeletal muscles (from 116.0±1.0 to 118.0±10.1 and from 105.7±4.9 to 110.5±1.1 number of vessel intersections for the EDL and TA, respectively). Chronic inhibition of 20-HETE formation using ABT also attenuated the increase in vessel density induced by electrical stimulation in skeletal muscle (from 111±7.4 to 121±4.35 and from 99.7±4.72 to 119.5±4.51, number of vessel intersections for the EDL and TA, respectively).

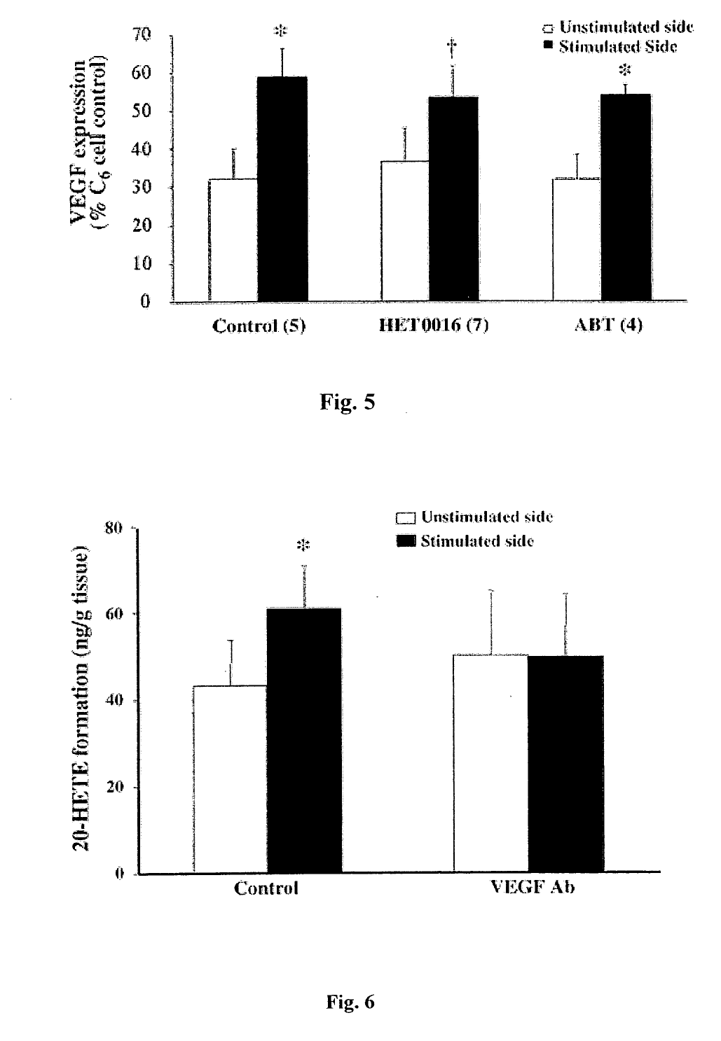

Because VEGF has been shown to play an important role in the angiogenesis of skeletal muscle, we performed Western blot analysis to verify the effects of HET0016 or ABT on VEGF protein expression. FIG. 5 shows the quantitative densitometry of the Western blot analysis used to compare the responses of VEGF protein expression after 7 days of stimulation in all of the animals treated with HET0016 or control. As shown in FIG. 5, VEGF protein levels were significantly increased by stimulation in control animals (P<0.05). To compare the effects of HET0016 with another CYP4A inhibitor, we treated a group of animals with ABT for 7 days during the electrical stimulation period, and the results are presented in FIG. 5. Neither HET0016 nor ABT had any effect on baseline VEGF expression. The increases in VEGF protein expression induced by electrical stimulation were not blocked by HET0016 or ABT.

In a complementary experiment, rats were treated with VEGF-neutralizing antibody or PBS (control) to analyze the role of VEGF on 20-HETE formation. As shown in FIG. 6, treatment with VEGF antibody completely blocked the increases in 20-HETE formation induced by 7 days of electrical stimulation.

### EXAMPLE 2

### Modulation of Growth Factor Induced Angiogenesis by 20-HETE in Rat Cornea

CYP4A enzymes metabolize arachidonic acid to 20-HETE. In this example, we demonstrate that 20-HETE is mitogenic in endothelial cells in vitro and angiogenic in vivo. We further demonstrate that the highly selective CYP4A inhibitor HET0016 blocks the mitogenic activity of VEGF in endothelial cells. DDMS is another selective inhibitor of CYP4A. We demonstrate that both HET0016 and DDMS inhibit angiogenic responses to VEGF in vivo. We further demonstrate that HET0016 blocks the angiogenic response to bFGF and EGF. We also demonstrate that HET0016 decreases angiogenesis induced by U251, a human glioblastoma cell line.

Materials and Methods

Reagents:

HET0016 [N-hydroxy-N′-(4-butyl-2 methylphenyl) formamidine] was synthesized as described in Miyata N et al. (Br J Pharmacol 2001, 133:325-9) and Sato M et al. (Bioorg Med Chem Lett 2001, 11:2993-5), both are incorporated by reference in their entirety, and was provided by Taisho Pharmaceuticals Corp (Satiama, Japan). The CYP4A inhibitor, DDMS and the stable 20-HETE agonist, WIT003[ 20-hydroxyeicosa-6(Z),15(Z)-dienoic acid], were synthesized by Dr. J R Faick of University of Texas Southwestern Medical Center (Capdevila J H and Falck J R, Prostaglandins Other Lipid Mediat 2002, 68-69:325-44) and have been used previously (Alonso-Galicia M et al., Am J Physiol 1999, 277:F790-6; Wang M H et al., J Pharmacol Exp Ther 1998, 284:966-73; and Yu M et al., Eur J Pharmacol 2004, 486:297-306). VEGF, bFGF and EGF were purchased from R&D Systems (Minneapolis, Minn.) and Hydron type NCC was obtained from Interferon (New Brunswick, N.J.). Primers for PCR were synthesized from Qiagen (Valencia, Calif.). Human umbilical vein endothelial cells (HUVECs) and related culture reagents were purchased from Cambrex (Walkerville, Md.). All other cell culture reagents were purchased from Invitrogen (Carlsbad, Calif.). Palmitic acid and all other reagents were purchased from Sigma Chemical Corp (St. Louis, Mo.).

Animals:

Experiments were performed in 7 to 8 week-old male Sprague-Dawley rats weighing 200-225 g (Charles River Laboratories, Wilmington, Mass.). Rats were housed in a 12 hr/12 hr light/dark cycle environment and provided with food and water ad libitum. All procedures complied with the ARVO statement for the use of animals in ophthalmic and vision research. Approval for the use of animals was obtained from the Institutional Animal Care and Use Committee (IACUC) of Henry Ford Health System, Detroit, Mich.

HUVECs Proliferation Assay:

HUVECs were seeded onto a 96-well plate at 1×10⁴ cells/well. Cultures were grown overnight and then exposed to either 10 μM HET0016, 1 μM WIT003, or 250 ng/ml VEGF alone or combined with either HET0016 or WIT003. HET0016 and WIT003 were both dissolved in ethanol. Organic solvent concentration never exceeded 0.1% of total culture volume. Cell Proliferation was measured 24 hours later using CellTiter96 AQueous One reagent (Promega, Madison, Wis.), a reliable colorimetric method for determining the number of viable cells in proliferation. 20 μl of Aqueous One reagent was added to the 100 μl medium in each well. The plates were incubated for 2 hours at 37° C. in a humidified incubator. Absorbance was recorded at 490 nm using a 96-well Bio Kinetics Reader EL340 (Bio-TEK, Winooski, Vt.). The data represent changes in percent absorbance of treated cultures compared with control cells. Three different experiments were carried out, and each point was determined in triplicate.

Cornea Pocket Angiogenesis Assay:

(i) Preparation of sustained-release polymer: Sustained-release polymer pellets were prepared by making a 1:1 mix of a 12% solution in ethanol of the polymer (Hydron polyhydroxyethylmethacrylate), with saline containing the growth factors. The growth factors bFGF, VEGF, and EGF were dissolved at a concentration of 125 ng/μl. HET0016 and WIT003 (Yu M et al., Eur J Pharmacol 2004, 486:297-306) were dissolved in ethanol to a concentration of 10 μg/μl while DDMS was dissolved in ethanol to a concentration of 5 μg/μl. 2 μl of these solutions were added to each pellet. Thus, a pellet containing 250 ng of a single growth factor was implanted at random in the right or left eye. The other eye was implanted with a pellet containing the same dose of growth factor+HET0016. In some rats the pellet contained VEGF alone and VEGF+DDMS. 20 μg of the stable 20-HETE agonist analog WIT003 was implanted in one eye to determine whether it induces angiogenesis. Since ethanol was the vehicle for HET0016, DDMS and WIT003, ethanol was added as a control to all other pellets. 9 μl of the 1:1 Hydron/treatment mixture was placed on the ends of 1.5-cm rods. Each pellet contained 250 ng of its respective growth factor. For bFGF we used sucralfate to stabilize and allow sustained release of this growth factor (Volkin D B et al., Biochim Biophys Acta 1993, 1203:18-26). After drying the pellets for 1 hour, they were ready to be implanted into the cornea of rats.

(ii) Pellet implantation: The rats were anesthetized IM with ketamine (80 mg/kg) and xylazine (10 mg/kg). The eyes were topically anesthetized with 0.5% proparacaine (Ophthetic, Alcon, Tex.) and the globes proptosed with a jeweler's forceps. Using an operating microscope, a central intrastromal linear keratotomy, approximately 1.5 mm in length, was performed with a surgical blade (Bard-Parker #11; Becton Dickinson, Franklin Lake, N.Y.) parallel to the insertion of the lateral rectus muscle. A curved iris spatula (No. 10093-13, Fine Science Tools, Belmont, Calif.) approximately 1.5 mm wide and 5 mm long was inserted under the lip of the incision and gently pushed through the stroma toward the temporal limbus of the eye. The distance between the limbus and the base of the pocket was kept at 1.0±0.1 mm. The pellet was advanced to the temporal end of the pocket. Antibiotic ointment (erythromycin) was applied to the anterior surface of the eye.

(iii) U251 human glioma cells spheroids: U251 human glioma cells were generously provided by Dr. Stephen Brown (Dept. of Radiation Oncology, Henry Ford Health System, Detroit, Mich.). The cells were maintained in DMEM (Invitrogen) supplemented with 10% heat-inactivated fetal bovine serum, penicillin (10 IU/ml), streptomycin (10 μg/ml) and 10% non-essential amino acids and grown at 37° C. in a humidified incubator containing 5% CO_{2.} U251 spheroids were obtained by a modification of the method of Carlsson and Yuhas (Carlsson J and Yuhas J M, Recent Results Cancer Res 1984, 95:1-23). Briefly, tumor cell spheroids were prepared by seeding a single-cell suspension (5×10⁶cells), over a layer of 0.8% Noble agar (Difco, Livonia, Mich.). Cells were grown for 2-3 days until spheroids formed. Spheroids of similar diameter were selected and then transferred onto a cell culture dish and washed with PBS to eliminate traces of serum. Spheroid diameter was measured using a dissection microscope equipped with a ruler. 5-8 spheroids, each approximately 200 μm in diameter, were aspirated into a syringe attached to blunt 27 gauge needle, and inserted into the corneal pocket. In this experiment, one eye contained the spheroids and a pellet containing ethanol (HET0016 solvent) while the other eye received spheroids and a pellet containing 20 μg HET0016.

(iv) Quantitation of corneal neovascularization: Seven days after pellet implantation, the rats were deeply anesthetized as previously described with ketamine and xylazine. The left ventricle was cannulated and the animal was perfused with 20-25 ml saline through the left ventricle followed by 20-25 ml India ink (waterproof drawing ink, Sanford, Bellwood, Ill.). The eyes were marked for orientation, enucleated and placed in 4% formalin for 24 hours. The cornea was dissected free from the surrounding globe and underlying iris, bisected, and loosely mounted between two glass slides, thus gently flattening the cornea. These flat mounts were examined microscopically using a Nikon Diaphot Epi-fluor 2 microscope attached to a CCD video camera and the images were digitized and saved using a computer.

Neovascularization was determined by comparing total vessel length in the control and experimental eyes. Vessel length was determined by tracing each vessel from the limbus to the pellet. Total length is the sum of these values in pixels and was determined using conventional image analysis software (Sigma Scan Pro, SPSS, Chicago, Ill.)

Groups:

In all cases pellets were implanted in both eyes. One eye served as control and the other was the experimental group. The following groups were studied.

Group 1. Controls.

Pellets containing only 2 μl ethanol were implanted in the rat corneas. In some experiments, we tested for nonspecific effects caused by the mere presence of fatty acids in the pellets. In these experiments, the pellet contained up to 40 μg palmitic acid (n=4). There was no difference in angiogenic responses to VEGF in eyes treated with only ethanol vs ethanol containing palmitic acid.

Group 2. Effects of the CYP4A Inhibitor HET0016

Control vs 20 μg HET0016 or 10 μg DDMS. These doses were chosen based on dose-response studies described below. (n=6). This group was included in order to determine whether the inhibitors had any visibly toxic or pro-angiogenic effects. In some rats, the effects of DDMS (10 μg/pellet) were also tested.

Group 3. Dose Response for HET0016 Inhibition of VEGF Angiogenic Responses

a. VEGF vs VEGF+5 μg HET0016 (n=4)

b VEGF vs VEGF+20 μg HET0016 (n=4)

c. VEGF vs VEGF+40 μg HET0016 (n=4)

Group 4. Anti Angiogenic Effects of HET0016.

In these rats we tested the effects of HET0016 in the neovascularization response to VEGF, bFGF and EGF.

a. VEGF vs VEGF+20 μg HET0016 (n=6)

b. bFGF vs bFGF+20 μg HET0016 (n=6)

c. EGF vs EGF+20 μg HET0016 (n=8).

Group 5. Anti Angiogenic Effects of a Second CYP4A Inhibitor.

In these rats we tested the effects of DDMS on the neovascularization response to VEGF.

VEGF vs VEGF+10 μg DDMS (n=7). This dose was selected after pilot experiments indicated it was as effective as 20 μg HET0016.

Group 6. Pro Angiogenic Effect of 20-HETE.

In these rats we tested whether the stable 20-HETE analog WIT003 was angiogenic.

Control vs 20 μg WIT003 (n=7)

Group 7. Anti Angiogenic Effects of HET0016.

In these rats, we studied HET0016 effects on cancer-induced angiogenic responses. For these experiments, we used the human glioblastoma cancer cell line U251, known to be angiogenic (Hsu S C et al., Cancer Res 1996, 56:5684-91). The spheroids and the pellet containing HET0016 were implanted together in the same cornea pocket. Angiogenic responses were assessed 14 days after implantation of the spheroids.

U251 cells spheroids vs U251 spheroids+20 μg HET0016 (n=8).

Cornea CYP4A1 mRNA Expression:

(i) mRNA extraction and cDNA synthesis: The expression of CYP4A1 mRNA in the cornea during neovascularization was determined using RT-PCR. The corneas were rapidly removed and snap-frozen in liquid nitrogen. The corneas were later thawed and homogenized in TRIzol. Total RNA was extracted from TRIzol according to the manufacturer's protocol (Invitrogen). Quality of the RNA was assessed by using the 260/280 nm absorbance ratio. Only samples within the 1.8-2.0 range were used. A total of 1-3 μg mRNA was reverse transcribed using the FirstStrand synthesis kit (Invitrogen). 1 μg of the cDNA was amplified by PCR.

(ii) PCR analysis: We amplified the CYP4A1 mRNA using the following specific CYP4A1 mRNA primers: Sense: TTCCAGGTTTGCACCAGACTCT (SEQ ID NO:1) and antisense: TTCCTCGCTCCTCCTGAGAAG (SEQ ID NO:2). Amplification of β-actin was used as an internal control. The primers were designed using Primer Express software from Applied Biosystems (Foster City, Calif.). The mRNA sequences of CYP4A1 were obtained from GeneBank with accession number NM_{—}175837 and for rat β-actin with accession number NM_{—}031144.

The PCR conditions used to amplify CYP4A1 and β-actin comprised a precycle of 95° C. for 3 min followed by 35 cycles consisting of 95° C. for 45 sec, 57° C. for 1 min, and 72° C. for 1 min, and then a final extension at 72° C. for 10 min. PCR products were subjected to electrophoresis in 10% acrylamide gels and visualized by ethidium bromide. Appropriate controls were used to ensure that amplified samples contained no genomic DNA.

Statistical analysis: The statistical significance of the differences in control and experimental eyes within rats was determined by paired t-test. The differences in responses between groups were determined by ANOVA followed by posthoc test. A p<0.05 was considered significant.

Results

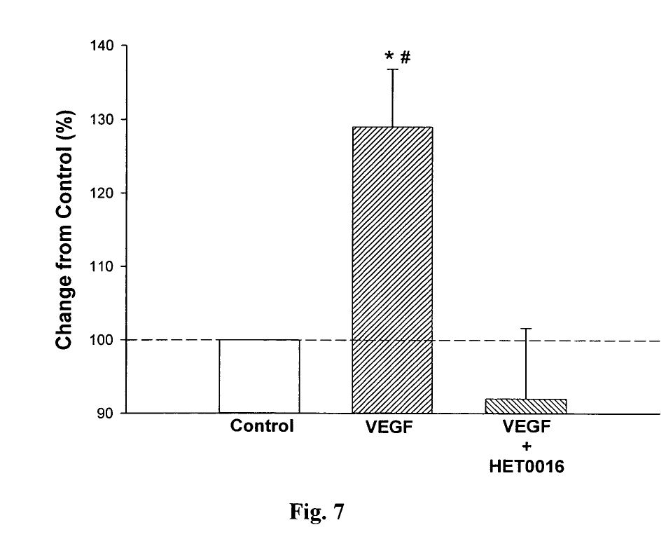

The effects of VEGF on the growth of HUVECs are presented in FIG. 7. VEGF stimulated proliferation in these cells, and this response was abolished in cultures simultaneously treated with HET0016. HET0016 did not alter basal proliferation rate of HUVECs (not shown).

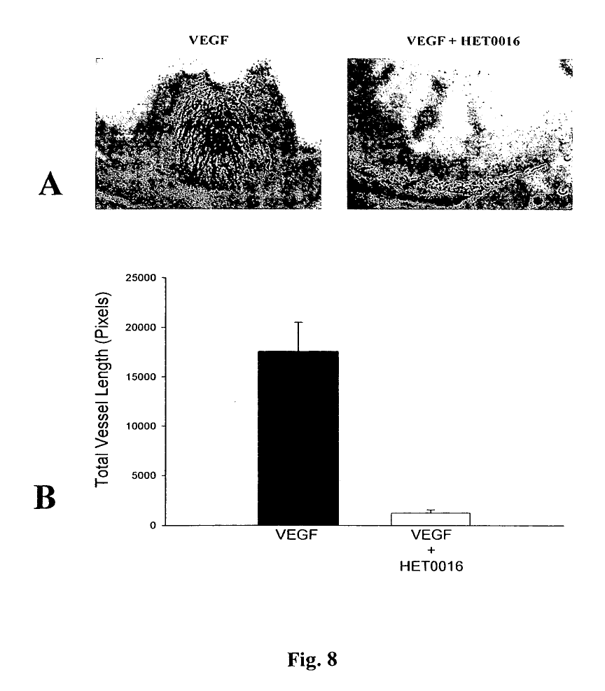

We next studied the effects of HET0016 on the angiogenic response to VEGF in vivo using the rat cornea pocket angiogenesis assay. Neither HET0016 nor DDMS alone elicited a response different from saline (FIGS. 8B and 11B). To determine the optimal dose of HET0016, we performed a dose-response study in which pellets containing different doses of HET0016 together with VEGF were implanted in the corneas. HET0016 at a dose of 20 μg and 40 μg/pellet almost completely abolished the angiogenic response to VEGF. 5 μg HET0016 reduced angiogenic responses to VEGF by about 50%. We therefore selected 20 μ/g per pellet of HET0016 or other related compounds, since all have comparable molecular weight and solubility. As a control for nonspecific effects of fatty acids, we used palmitic acid in some experiments. Palmitic acid had no effect by itself and showed no ability to influence angiogenic responses to VEGF or any of the other angiogenic factors studied (not shown).

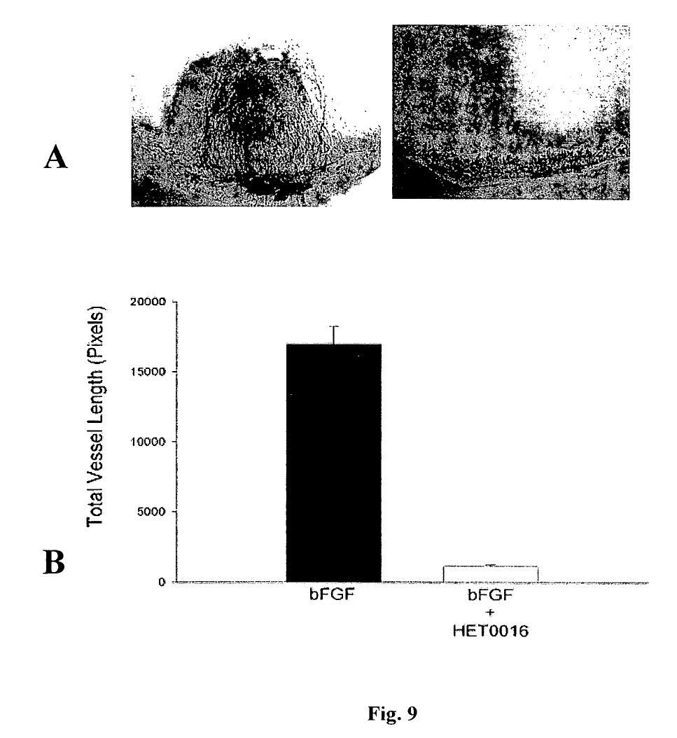

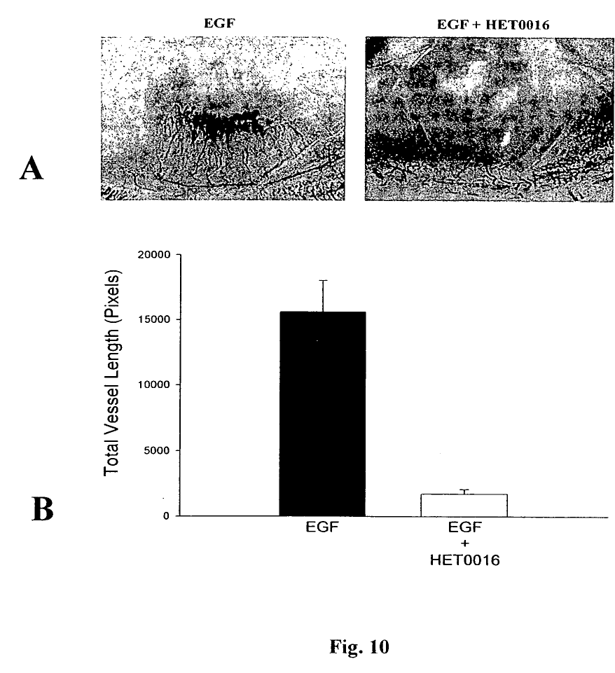

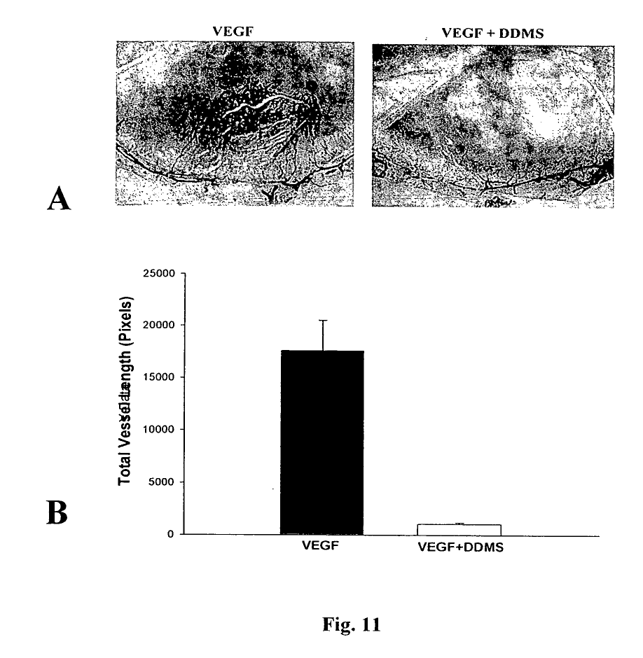

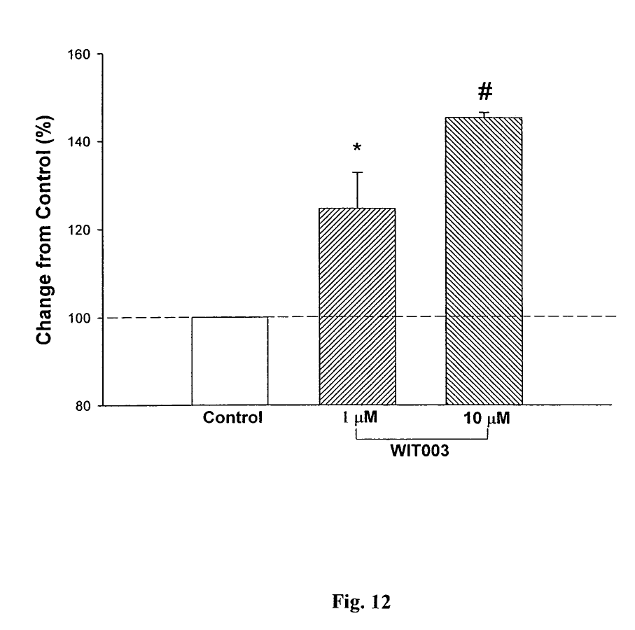

Inclusion of VEGF in the pellets resulted in a marked neovascularization response, which was obliterated by HET0016. FIG. 8A shows representative micrographs of corneas treated with VEGF alone and VEGF+HET0016, while FIG. 8B shows the results in graphic form. In other experiments, we examined the effect of blocking CYP4A activity on the angiogenic response to other growth factors. We studied whether the anti-angiogenic effect of HET0016 is constrained to VEGF or also suppresses responses to bFGF and EGF. Inclusion of HET0016 in the pellet drastically decreased the angiogenic response to both, bFGF (FIGS. 9A and 9B) and EGF (FIGS. 10A and 10B).

These data suggest that CYP4A activity is necessary for angiogenic growth factors to elicit an angiogenic response. To reinforce this concept, we tested whether a chemically dissimilar inhibitor of CYP4A, DDMS, also suppresses the angiogenic response to VEGF in the rat cornea pocket angiogenesis assay. The results of these experiments are presented in FIGS. 11A and 11B and indicate that DDMS also completely inhibits the angiogenic response to VEGF.

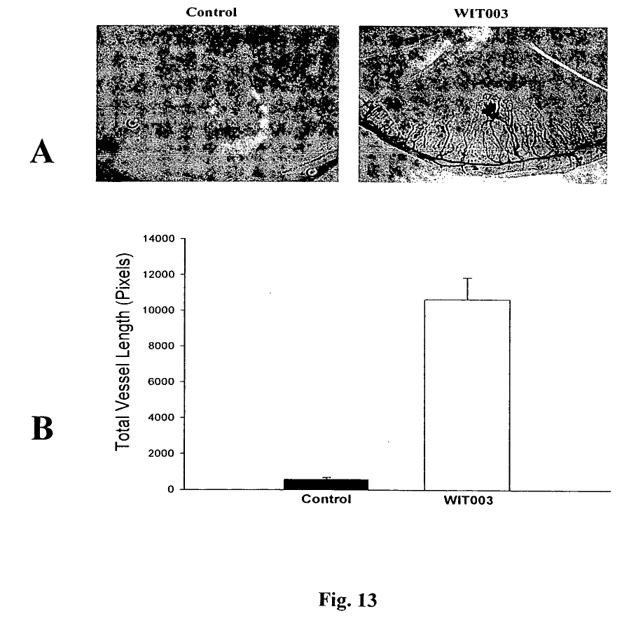

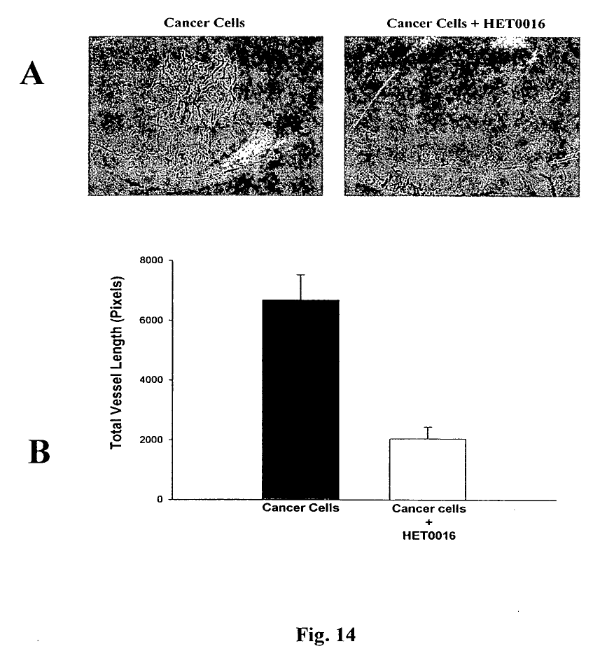

Since CYP4A is a ω-hydroxylase that synthesizes 20-HETE from arachidonic acid, HET0016 may act by inhibiting the synthesis of 20-HETE. To determine if 20-HETE may contribute to the anti-angiogenic activity of CYP4A inhibitors, we studied the effects of the stable 20-HETE analog WIT003 on the proliferation of HUVECs in vitro and the growth of new vessels in vivo. WIT003 increased the proliferation rate of HUVECs (FIG. 12), and induced an angiogenic response in the rat cornea pocket angiogenesis assay (FIGS. 13A and 13B),

We also studied whether HET0016 inhibited the angiogenesis induced by tumor cells. Three-dimensional spheroids of the human glioblastoma cell line U251 implanted in the cornea pocket led to marked angiogenesis after 2 weeks. Corneal neovascularization was significantly inhibited in the presence of HET0016 (p<0.01) (FIGS. 14A and 14B).

The results of the experiments to assess the expression of CYP4A mRNA in the rat corneas show that a band of the expected size was detected in control corneas. No detectable changes were observed in the VEGF-treated corneas.

In summary, the present study examined the effects of inhibitors of CYP4A on the mitogenic response of HUVECs to VEGF and in the growth factor-induced angiogenesis in the cornea in vivo. The results indicated that blockade of CYP4A activity with HET0016 blocked the known proliferative response to VEGF in HUVECs (Kurzen H et al., Inhibition of angiogenesis by non-toxic doses of temozolomide, Anticancer Drugs 2003, 14:515-22). Further evidence that a metabolite of arachidonic acid may play a role in VEGF-induced proliferation was suggested by experiments using the stable 20-HETE analog WIT003. Treatment of HUVECs with WIT003 increased proliferation of the cells in a manner similar to the changes following treatment of the endothelial cells with VEGF.

The angiogenic response in vivo involves much more than just an increase in endothelial cells proliferation, since other processes are involved including cell migration, matrix degradation, endothelial cell differentiation, and recruitment of perimural cells. Given the complexity of the angiogenic process, it is essential to demonstrate that any potential inhibitor of angiogenesis is able to affect the formation of fully formed blood vessels in vivo. Since we hypothesized that inhibition of CYP4A would alter angiogenic responses in vivo, we tested this hypothesis in the rat cornea pocket angiogenesis assay, an in vivo model of angiogenesis. This assay involves placing a pellet containing an angiogenic inducer (in our case angiogenic growth factors or cancer cells) into a pocket carved into the corneal stroma. The pellet slowly and continuously releases the angiogenic factor, and this in turn stimulates outgrowth from the peripherally located limbal vasculature toward the pellet, following the concentration gradient. In comparison to other in vivo assays, it has the advantage of measuring only new blood vessels, because the cornea is initially avascular and transparent (Kenyon B M et al., Invest. Ophthalmol. Vis. Sci. 1996, 37:1625-1632).

Vigorous angiogenic responses were observed when VEGF, bFGF, or EGF are implanted into the rat cornea. The angiogenic response to all of these growth factors was obliterated by the presence of HET0016. This suggests that CYP4A is a crucial regulator of angiogenesis, since angiogenic responses were practically abolished when a potent inhibitor of CYP4A was present. The olefinic compound DDMS has been reported to be a potent and highly selective inhibitor of CYP4A whose structure and mechanism of action is unrelated to that of HET0016 (Wang M H et al., J Pharmacol Exp Ther 1998, 284:966-73). The effects of VEGF on corneal neovascularization were also abolished by DDMS. Thus, two different inhibitors of CYP4A abolished the angiogenic responses induced by VEGF, consequently strengthening the concept that these inhibitors affect an essential step in the angiogenic process. Since inhibition of CYP4A is apparently the common link between HET0016 and DDMS, we conclude that one product of the enzymatic activity of CYP4A is either a mediator or a necessary component without which the angiogenic process does not proceed.

Angiogenesis is a crucial event in physiological conditions such as wound healing and the female reproductive cycle and also in pathological situations such as diabetic retinopathy, macular degeneration and chronic inflammatory diseases. In particular, tumor expansion is dependent on angiogenesis, which is critical for the growth of cancers above 1-2 mm. Consequently, suppressing a tumor's ability to generate new vessels is an appealing therapeutic target.

Therefore, we explored whether HET0016 would affect tumor-induced angiogenesis. For this we selected a malignant glioma cell model known to be highly angiogenic, the glioblastoma cell line U251. This is clinically relevant since glioblastoma multiforme is distinguished by intense angiogenesis (Hsu S C et al., Cancer Res 1996, 56:5684-91). We implanted U251 spheroids into the rat corneal stroma together with a pellet containing either HET0016 or control (palmitic acid or saline). All control eyes showed marked corneal neovascularization; however, HET0016 significantly decreased angiogenic responses by approximately 70%.

### EXAMPLE 3

### HET0016 Suppresses Cell Proliferation in Human Glioma Cancer Cells

This example shows that 20-HETE is important for the growth of human cancer cells. A stable 20-HETE agonist, 20-hydroxyeicosa-5(Z),14(Z)-dienoic acid, at 1 μM increased human glioma U251 cell proliferation by about 20%. Dose-response studies indicated that treatment with 10 μM HET0016 for 48 hr inhibited U251 cell proliferation by about 60%, associated with a 65% decrease in [³H]thymidine uptake. Dibromododecenyl methylsulfonimide (DDMS, also called N-methylsulfonyl-12,12-dibromododecyl-11-enamide), a structurally different inhibitor of CYP4A, also blocked cell proliferation by about 60%. Neither DDMS or HET0016 had any effect on the basal growth of normal human vascular endothelial cells or keratinocytes. Flow cytometry studies demonstrated that HET0016 specifically inhibits the proliferation of human U251 cancer cells by arresting the cell cycle at G₀/G₁. Adding the 20-HETE agonist (1 μM) reversed HET0016 blockade of cell proliferation by about 70%. Western blot experiments indicate that HET0016 alters tyrosine phosphorylation in U251 cancer cells, leading to inhibition which was seen at 24 and 48 hr after HET0016 treatment. Further studies showed that adding HET0016 specifically inhibited phosphorylation of p42/p44 MAPK and SAPK/JNK.

Materials and Methods

Cell lines and Reagents: U251 human glioma cells were obtained from Dr. Stephen L. Brown, Dept. of Radiation Oncology, Henry Ford Health System, Detroit, Mich. Human umbilical vein endothelial cells (HUVEC) were purchased from Cambrex (East Rutherford, N.J.). Primary human keratinocytes were obtained from Dr. George Murakawa, Dept. of Dermatology, Wayne State University, Detroit, Mich. HET0016 [N-hydroxy-N′-(4-butyl-2 methylphenyl) formamidine] was a gift from Taisho Pharmaceuticals, Japan. DDMS, palmitic acid, and EGF were purchased from Sigma (St. Louis, Mo.). WIT003[ 20-hydroxyeicosa-6(Z),15(Z)-dienoic acid], a 20-HETE agonist, was synthesized by Dr. John R. Falck, Department of Biochemistry, University of Texas Southwestern Medical Center, Dallas, Tex. All other cell culture reagents were purchased from Invitrogen (Carlsbad, Calif.).

Culture Conditions: Cells were routinely maintained in DMEM (Invitrogen) supplemented with 10% heat-inactivated fetal bovine serum (FBS), penicillin (10 IU/ml), streptomycin (10 μg/ml) and 10% non-essential amino acids (all purchased from Invitrogen). Cells were maintained at 37° C. in a humidified incubator containing 5% CO₂. They were grown in medium containing 10% FBS, which was then replaced with serum-free medium where U251 cells grow exponentially. Treatments were initiated one day after serum removal.

Cell Proliferation Assays: Proliferation studies were performed with cultures plated at a density that ensured exponential growth for at least 5 days. Growth medium was generally replaced by serum-free medium 24 hr after plating. Cells were treated with various concentrations of a given compounds for 24 or 48 hr as described in the Methods. HET0016, DDMS, and WIT003 were all dissolved and diluted in ethanol (EtOH). Organic solvent never exceeded 0.1% of total culture volume. Cells were harvested by exposure to 0.05% trypsin/EDTA and counted using a hemocytometer.

[³H]Thymidine Incorporation Studies: Thymidine incorporation studies were performed with cells grown in 35-mm culture dishes. Cultures were pulsed with [methyl-³H]thymidine (1 μCi/ml culture medium) at various times after treatment with HET0016 for 1 hr. Palmitic acid and EtOH served as fatty acid and vehicle controls, respectively. At the end of the pulse, the medium was aspirated and the cells rinsed twice with cold 1× phosphate buffered saline (PBS). The rinsed cultures were fixed by exposure to cold 5% trichloracetic acid overnight at 4° C., after which fixed cells were extracted as described previously (Scholler et al., Mol. Pharmacol. 45: 944-954, 1994). A second set of non-fixed dishes was treated with 0.05% trypsin/EDTA to estimate cell numbers. [³H]thymidine was detected by scintillation counting and expressed as dpm/10³ cells.

Flow Cytometry: Cells were cultured in 100-mm dishes at densities that ensured exponential growth at the time of harvest. The harvesting and processing protocols used to detect DNA by flow cytometry with propidium iodide (PI) have been described previously (Reiners et al., Carcinogenesis 20: 1561-1566, 1999). Cells were analyzed with a Becton Dickinson FACScan in the Wayne State University Flow Cytometry Core Facility, Detroit, Mich. Percentages of cells in the G₀/G₁, S, and G₂/M stages of the cell cycle were determined with a DNA histogram-fitting program (MODFIT; Verity Software, Topsham, Me.). A minimum of 10⁴ events/sample were collected.

DNA Fragmentation and TUNEL Assays: HET0016-treated cultures were washed twice with 1×PBS and incubated with lysis buffer [20 mM Tris-HCl, 10 mM EDTA, 0.3% Triton X-100]. Genomic DNA was extracted and separated on a 2% agarose gel. Separated DNA was visualized by staining gels with ethidium bromide (EtBr). At the same time, U251 cultures were seeded onto coverslips and treated with HET0016 for TUNEL assays. Coverslips were washed 3× with PBS and air-dried. Samples were then fixed with a freshly prepared fixation solution (4% paraformaldehyde in PBS, pH 7.4) for 1 hr at room temperature, followed by incubation in fresh permeabilization solution (0.1% Triton X-100 in 0.1% sodium citrate) for 2 min on ice. Finally, samples were processed using in situ Cell Death Detection Kit, AP (Roche Diagnostics, Indianapolis, Ind.) according to the manufacturer's recommendations.

RNA Isolation and Reverse Transcription—Polymerase Chain Reaction (RT-PCR): Cultures were treated with either 10 μM HET0016 or 100 μM DDMS for 24 and 48 hr, respectively. EtOH-treated cultures were used as solvent controls. Briefly, total RNA was isolated with Trizol reagent (Invitrogen) and 1-2 μg RNA was used to synthesize cDNA using a First Strand synthesis kit (Invitrogen). We used PCR primers that specifically recognize CYP4A11, and β-actin-specific primers. A 1 μCi ³²P per sample was added to Platinum PCR Supermix (Invitrogen). The PCR conditions used to amplify CYP4A 11 and β-actin consisted of a precycle of 95° C for 3 min followed by 35 cycles of 95° C for 45 s, 52° C. for 30 s, and 72° C. for 2 min, with final extension at 72° C. for 10 min. The primers used were: β-actin forward primer, 5′-TGC GTG ACA TTA AGG AGA AG-3′ (SEQ ID NO:3); β-actin reverse primer, 5′-GCT CGT AGC TCT TCT CCA-3′ (SEQ ID NO:4); CYP4A11 forward primer, 5′-CCA CCT GGA CCA GAG GCC CTA CAC CAC C-3′ (SEQ ID NO:5); CYP4A11 reverse primer, 5′-AGG ATA TGG GCA GAC AGG AA-3′(SEQ ID NO:6). PCR products were subjected to electrophoresis in 5% bis-acrylamide gels and visualized by autoradiography.

Nuclear Extract Preparation and Western Blotting: Cells were treated with 10 μM HET0016 for various times and washed twice with ice-cold 1×PBS. They were then pelleted by centrifugation at 1,000 g for 5 min at 4° C. Cells were lysed by adding RIPA buffer [20 mM HEPES (pH 7.4), 100 mM NaCl, 1% Nonidet P-40, 0.1% SDS, 1% deoxycholic acid, 10% glycerol, 1 mM EDTA, 1 mM NaVO₃, 50 mM NaF, and Protease Inhibitors Set 1 (Calbiochem, La Jolla, Calif.)]. Plates were then scraped and cells collected in a 1.5-ml centrifuge tube, followed by incubation on ice for 30 min. Homogenates of the cell suspension were centrifuged for 10 min at 14,000 g and 4° C.; the pellets were discarded and protein concentrations in the supernatant determined by bicinchoninic acid (BCA) protein assay.

Typically, 20 μg of protein was separated on a 14% Tris-glycine gel (Invitrogen) and electroblotted on a PVDF membrane (Biotrace, Bothell, Wash.). Membranes were blocked for 1 h at room temperature with blocking buffer [0.2% I-Block reagent (Tropix, Bedford, Mass.), 0.1% Tween-20 in 133 PBS] before incubation with primary antibodies in blocking buffer (overnight at 4° C.). Phospho-tyrosine (Y102), phospho-serine/threonine-Pro MPM2, phospho-p42/p44 MAPK (T202/Y204)(20G11), and phospho-SAPK/JNK(T183/Y185)(98F2) monoclonal antibodies were purchased from Upstate, Waltham, Mass. In addition, CYP4A polyclonal antibodies were purchased from both Research Diagnostics (Flanders, N.J.) and Chemicon (Ternecula, Calif.) to detect CYP4A proteins. Both phospho-tyrosine (Y102) and phospho-serine/threonine-Pro MPM2 were detected using a 1:2000 dilution of the corresponding antibodies, whereas phospho-p44/p42MAPK and phospho-SAPK/JNK were detected using 1:1000 antibody dilutions. CYP4A antibodies were used at 1:100 dilutions. After washing three times in washing buffer (1×TBS and 0.1% Tween-20), membranes were incubated for 1 hr at room temperature with a peroxidase-conjugated goat anti-mouse or anti-rabbit antibody (Upstate, Waltham, Mass.) (diluted 1:4000 in blocking buffer). Membranes were then washed 3× in washing buffer, and chemiluminescence detection was performed using an enhanced chemiluminescence kit (Upstate) according to the manufacturer's protocol. Actin was used as a loading control.

Statistical Analysis: Data were analyzed by the Tukey HSD test. The Statistica 5.0 software package (StaSoft, Tulsa, Okla.) was used to perform these calculations. Differences were considered statistically significant at p<0.05.

Results

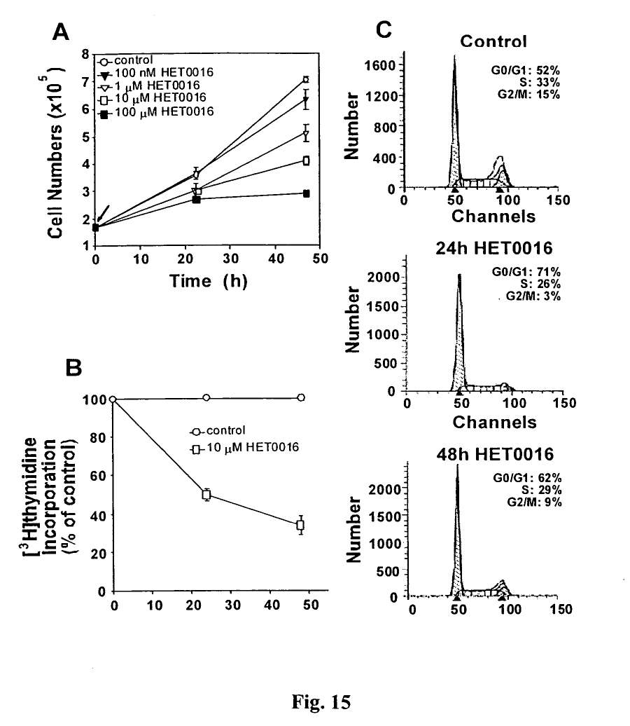

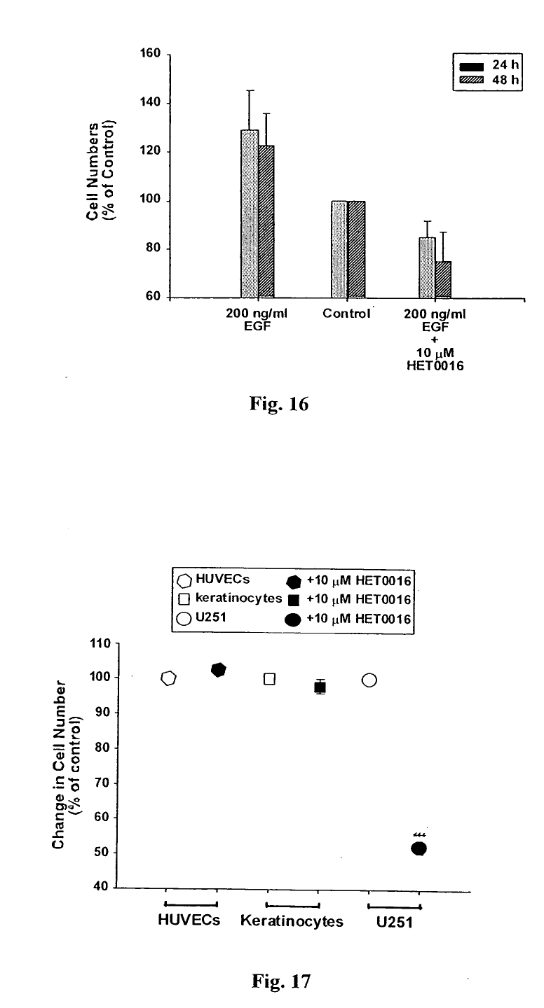

Effects of CYP4A Inhibition on Cell Proliferation: To assess the role of 20-HETE in regulating U251 cell growth, we studied the effects of the CYP4A inhibitor HET0016 on the proliferation and growth of human U251 glioma cancer cells in vitro. We treated cultures with various concentrations of HET0016 for 2 days and then counted the cells. Basal proliferation of U251 cells was suppressed by HET0016 in a concentration-dependent manner (FIG. 15A). This was considered a cytostatic effect, since trypan blue exclusion showed that the viability of the cells was not affected by HET0016. Furthermore, the proliferation induced by EGF was also inhibited by HET0016 (FIG. 16). Dose-response studies showed that 10 μM HET0016 inhibited proliferation of U251 cells by about 60% and this concentration was used as our working concentration for all subsequent studies. DNA fragmentation and TUNEL assays were performed to examine whether HET0016 induces apoptosis in U251 cells. Since both results were negative (data not shown), we concluded that HET0016 does not induce apoptosis in these cells. To determine whether HET0016 has the same effects on normal cells as it did on U251 cancer cells, we treated HUVECs and human primary keratinocytes with 10 μM HET0016. HET0016 had no effect on the basal growth or proliferation of either of these normal human cell types (FIG. 17).

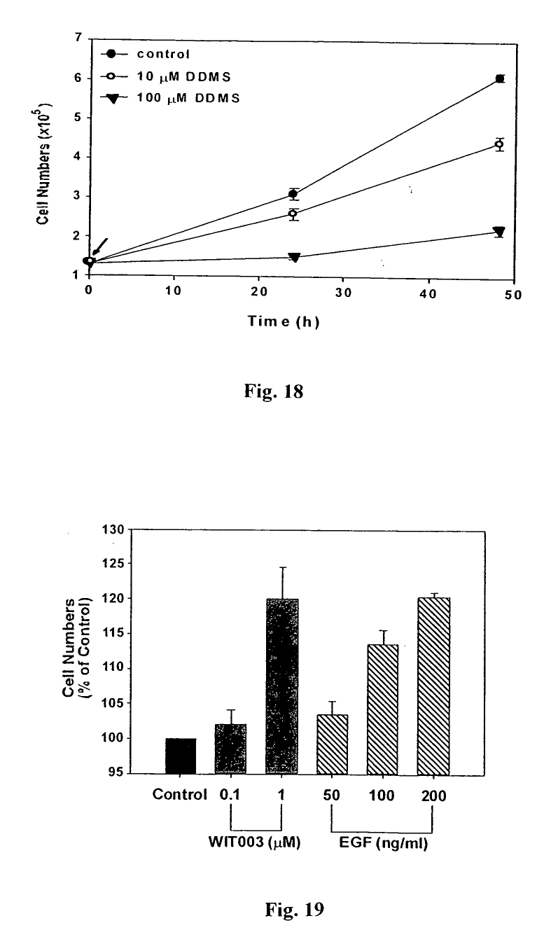

Analysis of [³H]thymidine incorporation indicated 50% and 66% inhibition of DNA synthesis approximately 24 and 48 hr after adding HET0016 to the culture media (FIG. 15B). DDMS, a structurally distinct inhibitor of the synthesis of 20-HETE, also inhibited the proliferation of U251 cancer cells in a dose-dependent manner (FIG. 18), similar to HET0016.

Flow Cytometry: Flow cytometry of cellular DNA content was performed to determine if the anti-proliferative effects of HET0016 reflected arrest at a specific point in the cell cycle (FIG. 15C). Cells containing G₀/G₁ DNA accumulated at 24 and 48 hr HET0016 treatment, accompanied by loss of both S and G₂/M phase cells.

CYP4A Expression at mRNA and Protein Levels: We performed RT-PCR and Western blot experiments to determine whether CYP4A is expressed in U251 cells. CYP4A11 mRNA was expressed in control U251 cultures as confirmed by DNA sequencing. Transcript levels were decreased after treating the cells with 10 μM HET0016 for 24 hr, whereas they were not affected in cultures treated with 100 μM DDMS. Thus CYP4A11 gene is transcribed and the message is expressed in U251 cultures. Additionally, Western blot experiments using two different CYP4A polyclonal antibodies obtained from two commercial sources showed that both antibodies detected immunoreactive proteins at about 55 kDa, consistent with the expected molecular weight for CYP4A. Thus, human U251 cancer cells express CYP4A at both the mRNA and protein levels.

Effects of 20-HETE Analog with Agonist activity on the Proliferation of U251 Cancer Cells: We tested the effects of a stable 20-HETE analog WIT003 with agonist properties on the proliferation of U251 cancer cells grown in culture. Serum-starved cultures were treated with 0.1 and 1.0 μM of 20-HETE agonist for 48 hr and the cells were subsequently counted. EGF was used as a positive control since it is known to produce maximal proliferation of U251 cells. A concentration of 0.1 μM WIT003 has no more stimulatory effect on proliferation of U251 cells than 50 ng/ml EGF, while at a concentration of 1.0 μM WIT003 stimulates the growth of U251 cancer cells by 20%. The magnitude of this effect is comparable to the growth stimulating effect induced by 200 ng/ml EGF (FIG. 19).

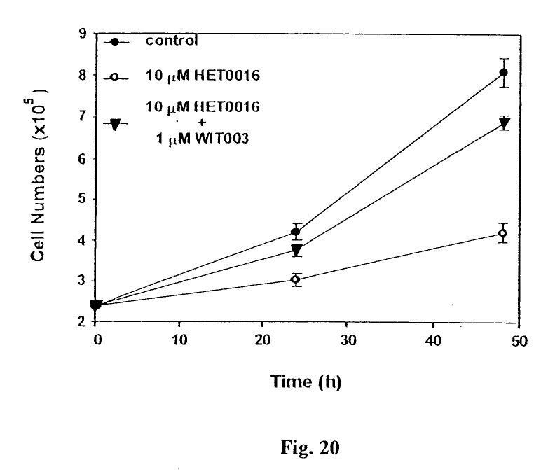

Reversal of the Anti-proliferative Effects of HET0016 by a 20-HETE Agonist: We examined whether exogenous addition of a 20-HETE agonist WIT003 can reverse the inhibition of the proliferation of U251 cells induced by blockade of the endogenous synthesis of 20-HETE with HET0016. In this experiment, U251 cultures were treated with 1 μM WIT003, 10 μM HET0016 or both compounds simultaneously. Cells were counted 2 days after treatment. In the presence of both WIT003 and HET0016, U251 proliferation increased to about 70% above that seen in cultures treated with HET0016 alone (FIG. 20). These findings indicate that addition of the stable 20-HETE agonist WIT003 reverses the effects of HET0016 to inhibit proliferation of U251 cancer cells.

Effects of CYP4A Inhibition on Phosphorylation of Signal Transduction Proteins in U251 Cells: To clarify the downstream signaling effects associated with inhibition of the formation of 20-HETE with HET0016 in U251 cells, protein extracts were isolated from U251 cultures treated with HET0016 for various times. To examine the changes induced by HET0016 in the tyrosine and serine/threonine protein phosphorylation status of proteins in U251 cells, Western blotting was performed using a phospho-tyrosine (Y102) antibody and a phospho-Ser/Thr-Pro MPM2 antibody. We found that HET0016 altered tyrosine phosphorylation in U251 cells, leading to a significant decreases at 24 and 48 hr after addition of HET0016. However, no significant changes in serine/threonine phosphorylation of proteins were seen in U251 cells treated with HET0016. Two additional antibodies were used to examine how HET0016 would affect phosphorylation of p42/p44 MAPK and SAPK/JNK. HET0016 inhibited both phosphorylation of p42/p44 MAPK and SAPK/JNK at 24 and 48 hr. Thus both MAPK and SAPK/JNK pathways can play a role in the signal transduction resulting from inhibition of CYP4A.

### EXAMPLE 4

### Inhibition of Rat Gliosarcoma Cell Proliferation by 20-HETE Inhibitors in Vitro and in Vivo

This example shows the effects of a selective inhibitor of CYP4A, HET0016 on the growth of 9L in vitro and in 9L-induced brain tumors in rats in vivo. CYP4A genes are highly expressed in 9L cells as determined by RT-PCR. Inhibition of CYP4A activity with the highly selective inhibitor, HET0016, decreased the proliferation of 9L cells in a dose-related manner. Addition of 10 μM HET0016 reduced the proliferation of 9L cells after 48 hrs by 60%. DDMS, a structurally different inhibitor of CYP4A enzymes also inhibited 9L cell proliferation by 60%. A stable 20-HETE agonist WIT003 at 1 μM rescued HET0016 inhibited cell proliferation by about 70%. EGF (200 ng/ml) increased the proliferation of 9L cells grown in vitro by about 30%. A similar degree of stimulation was obtained with 1 μM WIT003. HET0016 almost abolished EGF-induced growth of 9L cells. Western blot analysis showed that HET0016 decreases the phosphorylation of p42/p44 MAPK and SAPK/JNK. In rats, in vivo brain tumors were induced by injecting 9L cells directly in the forebrain. Treatment of rats with HET0016 (1 mg/Kg/day, ip) for about 2 weeks reduced by 80% the volume of the brain tumor. This was due to a reduction in mitosis of the injected 9L cells as well as increased apoptosis.

Materials and Methods

Culture Conditions: 9L rat gliosarcoma cells were purchased from ATC (Gaithersburg, Md.) and were maintained in DMEM (Invitrogen) supplemented with 10% heat-inactivated fetal bovine serum (FBS), penicillin (10 IU/ml), streptomycin (10 μg/ml) and 10% non-essential amino acids (all purchased from Invitrogen). Cells were maintained at 37° C. in a humidified incubator containing 5% CO₂. They were grown in medium containing 10% FBS, which was then replaced with serum-free medium (give type and source) where 9L cells grow exponentially.

Cell Proliferation Assays: Proliferation studies were performed with cultures plated at a density that ensured exponential growth for at least 5 days. Growth medium were generally replaced by serum free medium 24 hr after plating. Cells were treated with either HET0016 (Taisho Pharmaceuticals, Japan), DDMS, palmitic acid (a nonspecific fatty acid control), EGF (Sigma, St. Louis, Mo.), or WIT003 (a 20-HETE agonist) for 24 or 48 hr. HET0016, DDMS, and WIT003 were all dissolved and diluted in ethanol (EtOH). The concentration of the ethanol added to the media never exceeded 0.1%. Cells were harvested by exposure to a solution of 0.05% Trypsin/EDTA and counted using a hemocytometer.

[³H]Thymidine Incorporation Studies: Thymidine incorporation studies were performed with cells grown in 35-mm culture dishes. Cultures were pulsed with [methyl-³H]thymidine (1 μCi/ml culture medium) at various times after treatment with HET0016 for 1 hr. Palmitic acid and EtOH served as nonspecific fatty acid and vehicle controls, respectively. At the end of the pulse, the medium was aspirated and the cells rinsed twice with cold 1× phosphate buffered saline (PBS). The rinsed cultures were fixed by exposure to cold 5% trichloracetic acid overnight at 4° C., after which fixed cells were extracted as described previously (Scholler et al., Mol. Pharmacol. 45: 944-954, 1994). A second set of non-fixed dishes was treated with 0.05% trypsin/EDTA to estimate cell numbers. [³H]thymidine was detected by scintillation counting and expressed as dpm/10³ cells.

DNA Fragmentation and TUNEL Assays: HET0016-treated 9L cultures were washed twice with 1×PBS and incubated with lysis buffer [20 mM Tris-HCl, 10 mM EDTA, 0.3% Triton X-100]. Genomic DNA was extracted and separated on a 2% agarose gel. Separated DNA was visualized by staining gels with ethidium bromide (EtBr). At the same time, 9L cultures were seeded onto coverslips and treated with HET0016 for TUNEL assays. Coverslips were washed 3× with PBS and air-dried. Samples were then fixed with a freshly prepared fixation solution (4% paraformaldehyde in PBS, pH 7.4) for 1 hr at room temperature, followed by incubation in fresh permeabilization solution (0.1% Triton X-100 in 0.1% sodium citrate) for 2 min on ice. Finally, samples were processed using in situ Cell Death Detection Kit, AP (Roche Diagnostics, Indianapolis, Ind.) according to the manufacturer's recommendations.

RNA Isolation and Reverse Transcription—Polymerase Chain Reaction (RT-PCR): The cultures were treated with either 10 μM HET0016 or 100 μM DDMS for 48 hr. EtOH-treated cultures were used as solvent controls. Then, total RNA was isolated with Trizol reagent (Invitrogen) and 1-2 μg RNA was used to synthesize cDNA using a First Strand synthesis kit (Invitrogen). We used PCR primers that specifically recognize CYP4A1 and β-actin-specific primers. Platinum PCR Supermix (Invitrogen) was used as reaction mixture. The PCR conditions used to amplify CYP4A1/2/3 and β-actin consisted of a precycle of 95° C. for 3 min followed by 35 cycles of 95° C. for 45 s, 52° C for 30 s, and 72° C. for 2 min, with final extension at 72° C. for 10 min. The primers used were: β-actin forward primer, 5′-TTC AAC ACC CCA GCC ATG T-3′ (SEQ ID NO:3); β-actin reverse primer, 5′-GTG GTA CGA CCA GAG GCA TAC A-3′ (SEQ ID NO:4); CYP4A1/2/3 forward primer, 5′-TTC CAG GTT TGC ACC AGA CTC T-3′ (SEQ ID NO:5); CYP4A1/2/3 reverse primer, 5′-TTC CTC GCT CCT CCT GAG AAG-3′ (SEQ ID NO:6). PCR products were subjected to electrophoresis in 5% bis-acrylamide gels and visualized by autoradiography.

Nuclear Extract Preparation and Western Blotting: The 9L cells were treated with 10 μM HET0016 for various times and washed twice with ice-cold PBS. They were then pelleted by centrifugation at 1,000 g for 5 min at 4° C. The cells were lysed by adding RIPA buffer [20 mM HEPES (pH 7.4), 100 mM NaCl, 1% Nonidet P-40, 0.1% SDS, 1% deoxycholic acid, 10% glycerol, 1 mM EDTA, 1 mM NaVO₃, 50 mM NaF, and protease inhibitors Set 1 (Calbiochem, La Jolla, Calif.)]. The plates were then scraped and the cells were collected in a 1.5-ml centrifuge tube, followed by incubation on ice for 30 min. The cell homogenates were centrifuged for 10 min at 14,000 g and 4° C.; the pellets were discarded and the protein concentrations in the supernatant determined by bicinchoninic acid (BCA) protein assay.

Typically, 20 μg of protein obtained from the cell homogenates was separated on a 14% Tris-glycine gel (Invitrogen) and transferred to a PVDF membrane (Biotrace, Bothell, Wash.). The membranes were blocked for 1 hr at room temperature with blocking buffer [0.2% I-Block reagent (Tropix, Bedford, Mass.), 0.1% Tween-20 in 1×PBS] and then incubated with the primary antibodies in blocking buffer overnight at 4° C. Phospho-p42/p44 MAPK (T202/Y204)(20G11), and phospho-SAPK/JNK (T183/Y185) (98F2) monoclonal antibodies were purchased from Upstate (Waltham, Mass.). In addition, CYP4A polyclonal antibodies were purchased from Research Diagnostics (Flanders, N.J.) and Chemicon (Ternecula, Calif.) were used to detect CYP4A proteins. Both phospho-p44/p42MAPK and phospho-SAPK/JNK were detected using 1:1000 antibody dilutions. CYP4A antibodies were used at 1:100 dilutions. After washing the membranes three times in washing buffer (1×TBS and 0.1% Tween-20), the membranes were incubated for 1 hr at room temperature with a peroxidase-conjugated goat anti-mouse or anti-rabbit antibody (Upstate) (diluted 1:4000 in blocking buffer). The membranes were then washed 3× and developed using an enhanced chemiluminescence kit (Upstate). The membranes were then stripped and reprobed with a β actin primary antibody that served as a loading control.

Tumor Implantation: Before implantation, 90% confluent 9L cells were trypsinized and centrifuged. The cell pellet was resuspended in DMEM+10% FBS and counted using a hemocytometer. The concentration of the 9L cells was adjusted to 1×10⁴ cells/5 μl of medium. p The brain tumors were seeded by injecting the 9L cell suspensions into the frontal cerebral cortex of Fisher 344 rats that were purchased from Charles River Laboratories (Wilmington, Mass.) as follows. The rats were anesthetized with ketamine (80 mg/Kg, im) and Xylazine (13 mg/Kg), the head was immobilized in a stereotactic frame (David Kopf Instruments, Tujunga, Calif.) and the skull was exposed. A small hole was drilled in the skull 2 mm lateral and 2.5 mm anterior of the bregma, and 5 μl of the 9L gliosarcoma cell suspension were injected 3.5 mm into to cerebral cortex over a 5 min period using a 10 μl Hamilton (#2701) syringe attached to a 26 gauge needle. The hole was sealed with bone wax and the incisions were closed. The tumors were allowed to grow and become established for 2 days, then the rats received twice-daily sc injections of HET0016 or vehicle lecithin at a dose of 10 mg/kg/day. After 15 days of treatment with HET0016 or vehicle the rats were anesthetized with 80 mg/Kg and the brains were flushed with 250 ml of sterile 0.9% saline solution via cardiac puncture followed by perfusion fixation with 250 ml of 10% formalin in a physiological salt solution. The brains were removed and stored in 10% formalin.

Assessment of tumor volume: The formalin fixed brains were placed in Coronal rat brain matrix, and sliced into 3-mm blocks. These blocks were then embedded in paraffin and 6 μM thick sections were prepared. Sections prepared for H&E staining were placed onto uncoated slides. Sections for immunohistochemistry were placed onto positively charged super frosted slides. The serial sections were either stained with H&E for to assess the size of the tumor or immunohistochemically process for Ki-67 antigen to assess for the degree of proliferation.

Images of H&E stained sections containing tumor were captured using a SONY CCD camera using 2× objective. Using the AIS Image Analysis System (Imaging Research, St. Catherine, ON, Canada) software, the area of tumor in each section was manually outlined and measured in mm². The area was then multiplied by the section thickness to calculate a section volume. The volume of all the sections was then summed to obtain the total tumor volume for each rat.

The sections prepared for immunohistochemistry were deparaffinized by boiling the sections in a citric buffer (pH 6.0) for 10 minutes on a hot plate. The sections were cooled to room temperature, placed in a blocking buffer [2% normal serum, 1% BSA in PBS] for 1 hr, rinsed twice in a wash buffer [0.05 Tween-20 in PBS], blocked with hydrogen peroxide for 10 min and rinsed in wash buffer. The sections were then incubated for 30 min with rabbit polyclonal anti-ki67 antibody (Abcam Inc., Cambridge, Mass.; 1:200 dilution in 1.0% BSA in PBS). The sections were rinsed in wash buffer, incubated with biotinylated Goat anti-rabbit IgG (Vector Laboratories, Inc., Burlingame, Calif.; 1:500 in PBS) for 30 min, and rewashed. The sections were then incubated in HRP-streptavidin (Jackson ImmunoResearch Laboratories, Inc., West Grove, Pa.; 1:500 in PBS) for 30 min, and washed. DAB substrate (Vector Laboratories, Inc., Burlingame, Calif.; 2 drops of substrate buffer, 4 drops of DAB, 2 drops of peroxide in 5 ml of distilled water) was applied to the sections for 8 min. Sections were then washed, counterstained in Meyer's hematoxylin for 5 s, blued in ammonia water, rinsed in water, dehydrated, cleared, and mounted.

In Situ Apoptosis Analysis: Other sections were deparaffinized as described above and apoptosis analyzed using ApopTag peroxidase detection kit (Chemicon International Inc., Temecula, Calif.). Briefly, rehydrated sections were digested with proteinase K (20 μg/ml) for 15 min at room temperature followed by quenching in 3.0% H₂O₂ for 5 min and washed. Then, sections were labeled with a TdT enzyme in a humidified chamber at 37° C. for 1 hr and anti-digoxigenin conjugate were applied for 30 min at room temperature. Sections were washed, developed in peroxidase substrate, counterstained in 0.5% methyl green for 10 min, dehydrated, and mounted for light microscopy.

Statistical Analysis: Data were analyzed using and an ANOVA followed by a Tukeys test. Differences were considered statistically significant at P<0.05.

Results

RT-PCR for CYP4A: Since HET0016 is reportedly to be a highly selective inhibitor of the synthesis of 20-HETE catalyzed by CYP4A and 4F enzymes, we first examined whether CYP4A mRNA is expressed in 9L gliosarcoma cells by RT-PCR. We observed that mRNA for CYP4A is expressed in 9L cells grown in vitro.

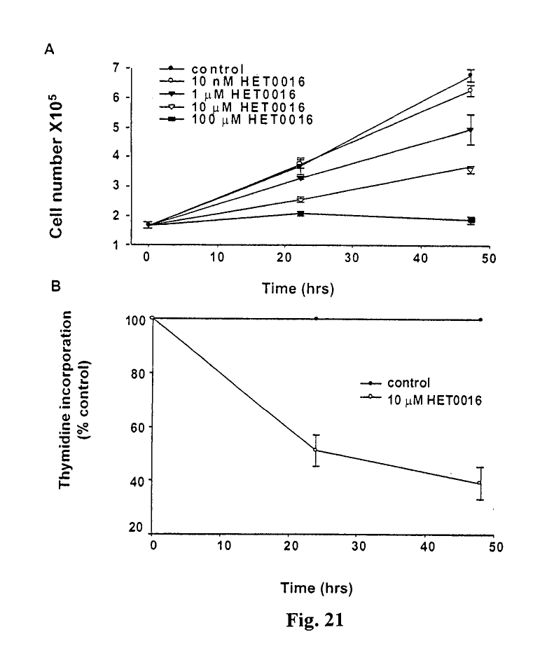

Effects of CYP4A Inhibition on the Proliferation of 9L Gliosarcoma Cells In Vitro: The effects of various concentrations of HET016 on the growth of 9L gliosarcoma cells is presented in FIG. 21. HET0016 produced a dose dependent inhibition of the growth of 9L cells grown in culture as directly assessed by cell counting (FIG. 21A). Even at a very low concentration of 10 nM which is near the reported IC50 of this compound to inhibit some isoforms of CYP4A some degree of inhibition of cell growth was apparent even though the difference from control was not significant (p=0.056). HET0016 at concentration of 1 and 10 μM clearly reduced cell number at both 24 and 48 hr by 30-40%.

In additional experiments we tested the effects of a high concentration (100 μM) of HET0016 on the growth of 9L cells. This produced a dramatic reduction in cell number. However, we also observed substantial cell detachment and dead cells floating in the media suggesting that at this concentration HET0016 may have a direct cytotoxic effect. Thus, we decided to use HET0016 at a concentration of 10 μM in all subsequent experiments. As is presented in FIG. 21B, HET0016 (10 μM) reduced thymidine incorporation in cultures of 9L gliosarcoma cells by 60%. Examination of the growth curves presented in FIG. 21B indicates that HET0016 alters the slope of this relationship indicating that it effects proliferation of cells rather than killing a population of cells and causing an expected baseline shift in the relationship.

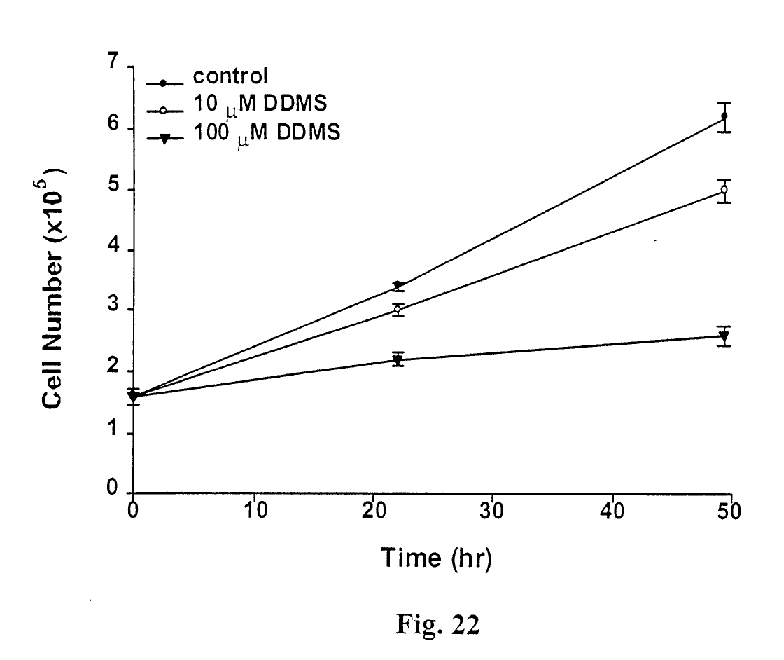

Effects of DDMS on the Proliferation of 9L Cells In Vitro: DDMS is a highly selective suicide substrate inhibitor of CYP4A but with a chemical structure and mechanism of action that is very different from HET0016. The effect of various concentrations of DDMS on the growth rate of 9L cells in vitro is presented in FIG. 22. The results are comparable with those observed with HET0016. At a concentration of 10 μM, which is near the IC50 for inhibition of CYP4A enzyme activity, DDMS produced a significant reduction in cell number. At a higher concentrations (100 μM), DDMS had a similar effect as HET0016 (10 μM) to reduce the proliferation of these cells.

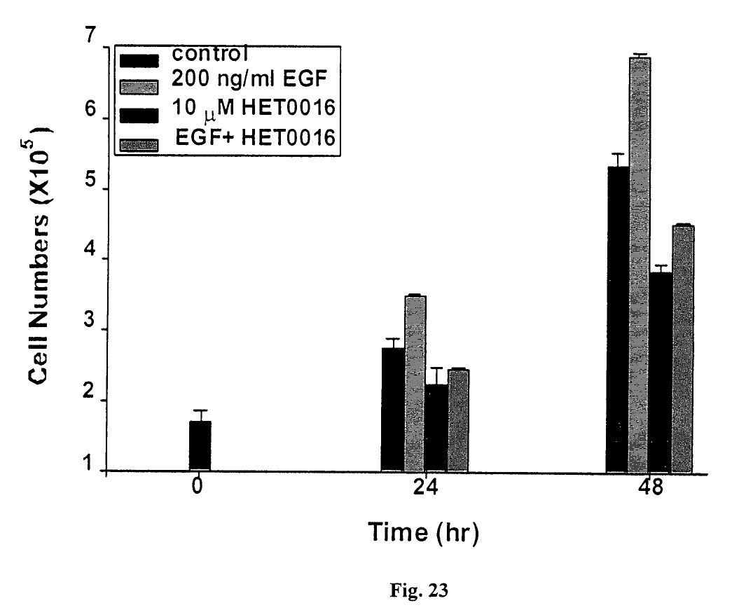

Effects of HET0016 on the EGF Stimulated Growth of 9L Cells In Vitro: Exposure of 9L cells to EGF (200 ng/ml) increased the number of cells at both 24 and 48 hrs (FIG. 23). Addition of HET0016 (10 μM) prevented the effects of EGF on cells growth at 24 hr and reduced the number of cells at 48 hr (FIG. 23). A comparison of the slopes of the growth curves suggests that the inhibitory effects of HET0016 waned between 24 and 48 hrs.

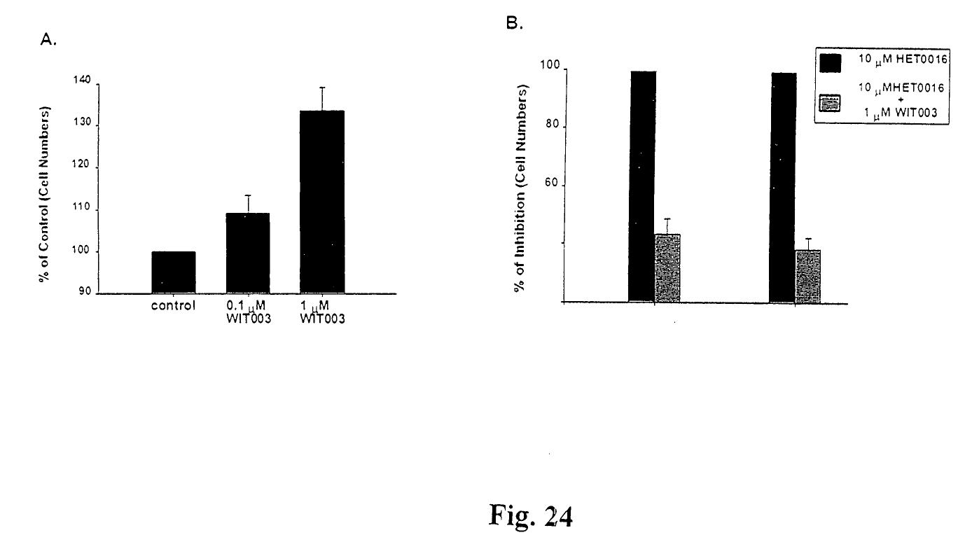

Effects of a 20-HETE Analog on the Growth Inhibitory Actions of HET0016: To determine whether the antiproliferative effects of HET0016 on the growth of 9L gliosarcoma cells were related to inhibition of the synthesis of 20-HETE we examined whether exogenous additions of WIT003, a stable 20-HETE agonist, would prevent the antiproliferative actions of HET0016. The results presented in FIG. 24 indicate that addition of WIT003 (1 μM) to the culture media partially rescued the 9L cells from the inhibitory actions of HET0016. Considering the inhibition induced by HET0016 alone as 100%, cells treated with HET0016+WIT003 show only a 60% improvement in proliferation

Effects of HET0016 on Tyrosine Phosphorylation of MAPK and JNK: To advance the understanding of the mechanism by which HET0016 may inhibit the growth of 9L cells, we examined the effects of HET0016 on the phosphorylation and activation of Mitogen-activated protein kinases (MAPK), known to play a key role in the regulation of cell growth and proliferation in 9L cells. Treatment of the 9L cells with HET0016 (10 μM) for 4 hr significantly reduced the phosphorylation of both p42/p44 MAPK and SAPK/JNK. The reduction in the phosphorylation of p42/p44 MAPK was even greater after 24 hr exposure to HET0016. The same trend was observed for the phosphorylation of JNK, although the inhibition appears to peak at 48 hr rather than 24 hr of exposure to HET0016.

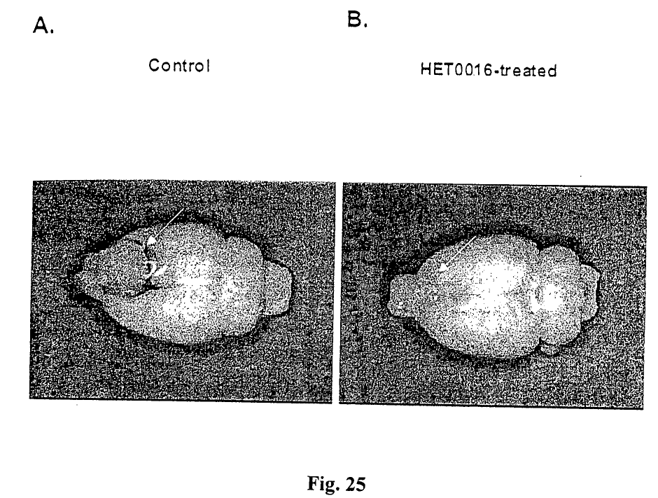

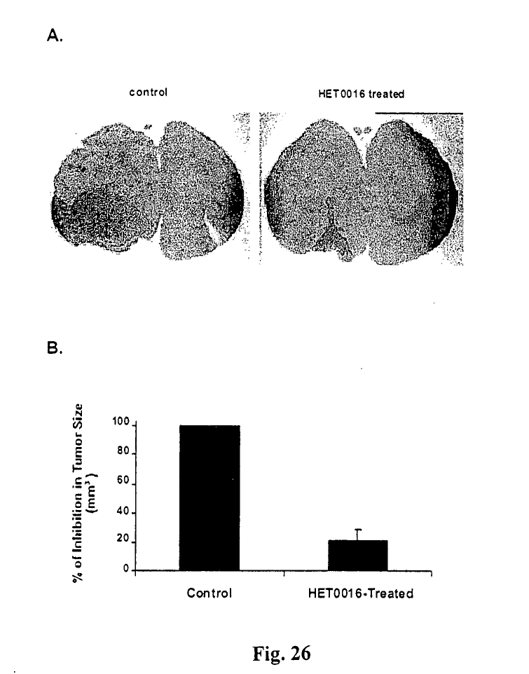

Effects of CYP4A and 4F Inhibitors on the Growth of Rat 9L Gliosarcoma Brain Tumors in Rats In Vivo: After 9L cells are implanted into the forebrain of normal, immunocompetent rats, they form a rapidly growing tumor with defined borders. Under the present experimental conditions, death of the animal usual occurs after 2 and 3 weeks if left untreated. In the present study 17 days after implantation of the tumor, we found that rats treated with HET0016 looked healthier and exhibited a much smaller tumor at necropsy than that seen in untreated control rats (FIG. 25). A representative section through the midplane of the tumors in control and HET0016 treated rats along with a comparison of the volume of the tumors are presented in FIG. 26. The volume of the tumor in the HET0016-treated rats was reduced by 80% compared to those seen in control animals (FIG. 26).

Results of in Situ Apoptosis Analysis: Sections of 9L tumors were immunostained with antibodies to determine areas of cell proliferation and apotosis. Chronic treatment of the rats with HET0016 greatly reduced the number of mitotic cells in the 9L tumor that stain positive with the Ki67 antibody. In contrast, there was not significant difference in the number of apoptotic cells in the 9L tumor that stained positive in rats treated with vehicle or HET0016. These results indicate that inhibition of the synthesis of 20-HETE with HET0016 limits the growth of the tumors by arresting cell proliferation rather than stimulating apotosis and the programmed death of the cancer cells.

The present invention is not intended to be limited to the foregoing examples, but encompasses all such modifications and variations as come within the scope of the appended claims.

1. A method for reducing angiogenesis in a tissue of a human or non-human mammal comprising the step of:
administering an agent selected from a 20-HETE synthesis inhibitor or a 20-HETE antagonist to the human or non-human mammal in an amount sufficient to reduce angiogenesis in the tissue.

2. The method of claim 1, wherein the agent is a 20-HETE synthesis inhibitor.

3. The method of claim 2, wherein the 20-HETE synthesis inhibitor is selected from N-hydroxy-N′-(4-butyl-2-methylphenol)-formamidine (HET0016), dibromododecenyl methylsulfonimide (DDMS), N-(3-Chloro-4-morpholin-4-yl)phenyl-N′-hydroxyimidoformamide (TS-011), 1-aminobenzotriazole (ABT), 17-Octadecynoic acid (17-ODYA), ketoconazole, miconazole, fluconazole, or 10 undecynyl sulfate (10-SUYS).

4. The method of claim 3, wherein the 20-HETE synthesis inhibitor is HET0016 or TS-011.

5. The method of claim 1, wherein the agent is a 20-HETE antagonist.

6. The method of claim 1, wherein the method is used to reduce angiogenesis induced by a growth factor.

7. The method of claim 6, wherein the growth factor is selected from vascular endothelial growth factor (VEGF), basic fibroblast growth factor (bFGF), and epidermal growth factor (EGF).

8. The method of claim 1, wherein the method is used to reduce angiogenesis induced by cancer or tumor cells.

9. The method of claim 1, wherein the method is used to reduce angiogenesis in a non-muscle tissue.

10. The method of claim 1, wherein the method is used to treat or prevent a disease or condition associated with abnormal, excessive blood vessel development in the human or non-human mammal.

11. The method of claim 10, wherein the disease is cancer.

12. The method of claim 10, wherein the disease is an eye disease associated with abnormal, excessive blood vessel development.

13. A method for reducing angiogenesis in a tissue of a human or non-human mammal comprising the step of:
administering N-(3-Chloro-4-morpholin-4-yl)phenyl-N′-hydroxyimidoformamide (TS-011), N-hydroxy-N′-(4-butyl-2-methylphenol)-formamidine (HET0016), or dibromododecenyl methylsulfonimide (DDMS) to the mammal in an amount sufficient to reduce angiogenesis in the tissue.

14. A method for inducing and promoting angiogenesis in a tissue of a human or non-human mammal comprising the step of:
administering an agent selected from 20-HETE or a 20-HETE agonist to the human or non-human mammal in an amount sufficient to promote angiogenesis in the tissue.

15. The method of claim 14, wherein the agent is a 20-HETE agonist.

16. The method of claim 14, wherein the method is used to promote angiogenesis in a non-muscle tissue.

17. The method of claim 14, wherein the method is used to treat or prevent a disease or condition associated with insufficient blood vessel development or vessel regression in the human or non-human mammal.

18. A method for inducing and promoting angiogenesis in a tissue of a human or non-human mammal comprising the step of:
administering 20 hydroxyeicosa-6(Z),15(Z)-dienoic acid to the mammal in an amount sufficient to induce and promote angiogenesis in the tissue.

19. A method for inhibiting cancer or tumor cell proliferation comprising the step of:
exposing cancer or tumor cells to an agent selected from a 20-HETE synthesis inhibitor or a 20-HETE antagonist in an amount sufficient to inhibit proliferation of the cancer or tumor cells.

20. The method of claim 19, wherein the agent is a 20-HETE synthesis inhibitor.

21. The method of claim 20, wherein the 20-HETE synthesis inhibitor is selected from N-hydroxy-N′-(4-butyl-2-methylphenol)-formamidine (HET0016), dibromododecenyl methylsulfonimide (DDMS), N-(3-Chloro-4-morpholin-4-yl)phenyl-N′-hydroxyimidoformamide (TS-011), 1-aminobenzotriazole (ABT), 17-Octadecynoic acid (17-ODYA), ketoconazole, miconazole, fluconazole, or 10 undecynyl sulfate (10-SUYS).

22. The method of claim 21, wherein the 20-HETE synthesis inhibitor is HET0016 or TS-011.

23. The method of claim 19, wherein the agent is a 20-HETE antagonist.

24. The method of claim 19, wherein the cancer or tumor cells are human or rat glioma cells.

25. The method of claim 19, wherein the method is used to treat or prevent cancer or tumor in a human or non-human mammal by administering the agent into the human or non-human mammal.

26. The method of claim 25, wherein the cancer is selected from glioma, astrocytoma, intestinal carcinoma, breast carcinoma, skin carcinoma, lung carcinoma, stomach carcinoma, prostate carcinoma, thyroid carcinoma, liver carcinoma, pancreatic carcinoma, kidney carcinoma, colon carcinoma, or ovarian carcinoma.

27. The method of claim 26, wherein the cancer is selected from glioma, breast carcinoma, skin carcinoma, prostate carcinoma, pancreatic carcinoma, or colon carcinoma.

28. A method for inhibiting tumor or cancer cell proliferation comprising the step of:
exposing tumor or cancer cells to N-(3-Chloro-4-morpholin-4-yl)phenyl-N′-hydroxyimidoformamide (TS-011), N-hydroxy-N′-(4-butyl-2-methylphenol)-formamidine (HET0016), or dibromododecenyl methylsulfonimide (DDMS) in an amount sufficient to inhibit proliferation of the tumor or cancer cells.


SEQUENCE LISTING (6 sequences)

SEQ ID NO: 1 - DNA, 22 bp, Artificial
  synthetic PCR primer
ttccaggtttgcaccagactct

SEQ ID NO: 2 - DNA, 21 bp, Artificial
  synthetic PCR primer
ttcctcgctcctcctgagaag

SEQ ID NO: 3 - DNA, 20 bp, Artificial
  synthetic PCR primer
tgcgtgacattaaggagaag

SEQ ID NO: 4 - DNA, 18 bp, Artificial
  synthetic PCR primer
gctcgtagctcttctcca

SEQ ID NO: 5 - DNA, 28 bp, Artificial
  synthetic PCR primer
ccacctggaccagaggccctacaccacc

SEQ ID NO: 6 - DNA, 20 bp, Artificial
  synthetic PCR primer
aggatatgggcagacaggaa